(19) United States
(12) Patent Application Publication
(10) Pub. No.: US 2007/0010444 A1
(43) Pub. Date: Jan. 11, 2007

(54) Novel mediator of neurotransmitter and psychostimulant responses

(75) Inventors: Miles Trupp, Greenbrae, CA (US); Steven L. McIntire, Tiburon, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/442,664

(22) Filed: May 26, 2006

Related U.S. Application Data
(60) Provisional application No. 60/686,226, filed on May 31, 2005.

(51) Int. Cl. A61K 38/17; C07K 14/705; C12Q 1/68; C07H 21/04; C12P 21/06

(52) U.S. Cl. 514/12; 435/6; 435/69.1; 435/320.1; 435/325; 530/350; 536/23.5

(57) ABSTRACT

The present invention provides polypeptides (termed “egl-28 polypeptides”) that modulate neurotransmitter- and psychostimulant-induced responses. The invention also provides methods of modulating such responses, as well as and anti-egl-28 antibodies. The invention encompasses related polynucleotides, vectors, host cells, production methods, and compositions. Moreover, the invention includes methods for prescreening or screening for test agents that modulate neurotransmitter and psychostimulant responses.

### CROSS-REFERENCE TO RELATED APPLICATIONS

This application claims priority to and benefit of U.S. Ser. No. 60/686,226, Filed May 31, 2005, which is incorporated herein by reference in its entirety for all purposes.

### STATEMENT AS TO RIGHTS TO INVENTIONS MADE UNDER FEDERALLY SPONSORED RESEARCH AND DEVELOPMENT

Not Applicable.

### FIELD OF THE INVENTION

The present invention relates to generally to a family of novel mediators of amphetamine and neurotransmitter responses and related methods and compositions. The novel mediators belong to the egl-28 gene family.

### BACKGROUND OF THE INVENTION

Psychostimulants such as amphetamine are widely used and abused drugs. Amphetamine and related compounds elicit multiple physiological effects, but are most widely recognized for their neurostimulatory action. Users report increased alertness, decreased fatigue, mood elevation and increased initiative. Performance of certain mental and physical tasks may be improved, possibly explaining the frequency of abuse among athletes. Psychostimulants have proven to be useful therapeutics in the treatment of a variety of conditions including ADHD (attention deficit hyperactivity disorder), obesity, narcolepsy and respiratory ailments (1). However, the therapeutic usefulness of these compounds is limited by additional neurotoxic affects observed with increased dosage or prolonged use (2). These include confusion, hallucinations, anxiety, hypertension and cardiac arrythmias (1). Prolonged use of psychostimulants is also complicated by the development of drug dependency or addiction as well as withdrawal symptoms upon termination of drug use (3).

Many of the effects of psychostimulants appear to be due to the inhibition of uptake of monoamine neurotransmitters from the extracellular space through an interaction of the drug with plasma membrane neurotransmitter transporters (4, 5). The reinforcing or addictive properties of these drugs have generally been attributed to blockade of the dopamine transporter, DAT (9). However, DAT-KO mice have been reported to continue to self-administer cocaine (6). The DAT-KO mice also exhibit hyperlocomotion in the absence of amphetamine and cocaine (7), and a paradoxical slowing of locomotion in response to amphetamine and cocaine (8, 9). This contrasts with the normal activating effect of these drugs in wild-type mice, but may relate to the recognized calming effect that these drugs have on individuals with hyperkinetic disorders such as ADHD (10). Some of the behavioral responses that persist in the DAT-KO mice have been attributed to an effect of amphetamine on other monoamine transporters such as the serotonin transporter, SERT. It is also possible that additional mechanisms, other than inhibition of monoamine transport, contribute to some of the behavioral effects of these drugs. Biochemical studies have also revealed multiple amphetamine binding sites in intact brain tissues and brain membrane preparations (11). It is unknown whether or not these binding sites correspond to behaviorally relevant drug targets.

Genetic screening for mutants exhibiting altered drug responses, such as drug resistance or hypersensitivity, provides an alternative means of identifying additional mechanisms of action of a drug. By characterizing the mutants, novel genes that are necessary for drug responses can be identified and may represent additional drug targets. One advantage of this approach is that no a priori assumptions are made regarding the nature of the molecular targets.

### SUMMARY OF THE INVENTION

An additional target of amphetamine was identified by screening for amphetamine resistant C. elegans mutants. One of the amphetamine resistant mutants obtained has a mutation in egl-28. egl-28 encodes a multi-pass transmembrane protein that is related to the nrf-6 family of proteins in C. elegans. The results of this work indicate that mechanisms other than modulation of monoamine transport contribute to the neuropharmacological effects of amphetamine.

Accordingly, the invention provides a method of modulating a response to a neurotransmitter or a psychostimulant. The method entails contacting cells expressing a member of the egl-28 gene family, with an effective amount of a modulator of the member of the egl-28 gene family, in the presence of the neurotransmitter or psychostimulant, respectively. An exemplary neurotransmitter useful in the method of the invention is histamine, and an exemplary psychostimulant is amphetamine. In particular embodiments, the modulator includes an inhibitor of the member of the egl-28 gene family. This method can be carried out using cells in vitro or in vivo.

Generally, the member of the egl-28 gene family includes a polypeptide including an amino acid sequence that has at least about 50% identity to the C. elegans egl-28 amino acid sequence (SEQ ID NO:3) over a comparison window of at least 17 contiguous amino acids. Aternatively, or in addition, the amino acid sequence can have at least about 20% identity to the full-length the C. elegans egl-28 amino acid sequence (SEQ ID NO:3).

Another aspect of the invention is an isolated polypeptide including an amino acid sequence that has at least about 50% identity to the C. elegans egl-28 amino acid sequence (SEQ ID NO:3) over a comparison window of at least 17 contiguous amino acids, provided the polypeptide is not nfr-6 or ndg-4. Aternatively, or in addition, the amino acid sequence can have at least about 20% identity to the full-length the C. elegans egl-28 amino acid sequence (SEQ ID NO:3). In an exemplary embodiment, the amino acid sequence includes the amino acid sequence of C. elegans egl-28 (SEQ ID NO:3). The amino acid sequence can be a vertebrate sequence, and in particular, a human sequence, e.g., the human ortholog (SEQ ID NO: 6) of C. elegans egl-28. In certain embodiments, the amino acid sequence defines a multi-pass transmembrane protein. The polypeptide of the invention can include other amino acid sequences, such as, for example, an epitope tag.

The invention also provides an isolated polynucleotide that encodes the polypeptide of the invention. The polynucleotide can include other nucleotide sequences as well, such as a sequence encoding a heterologous signal sequence or an epitope tag. Also within the scope of the invention are vectors including any of the polynucleotides of the invention. In one embodiment, the vector is an expression vector. Additionally, the invention includes host cells including any vector of the invention. Where the vector is an expression vector, a host cell containing it can be cultured under conditions suitable for expression of the encoded polypeptide; and the expressed polypeptide can be recovered from the culture.

In another aspect, the invention provides an antibody or antiserum that specifically binds to a polypeptide of the invention. In particular embodiments, the antibody or antiserum is produced by a method that entails administering the polypeptide or a fragment thereof to a mammal, and recovering the antiserum or cells producing the antibody from the mammal.

The invention also encompasses a method of identifying an egl-28 ortholog. The method entails determining whether a candidate egl-28 ortholog polynucleotide includes a nucleotide sequence that is substantially similar to an egl-28 nucleotide sequence and/or whether a candidate egl-28 ortholog polypeptide includes an amino acid sequence that is substantially similar to an egl-28 amino acid sequence. The ortholog identified can be a vertebrate ortholog, and in particular, a human ortholog.

Candidate othologs can, according to the invention, be subjected to a variety of manipulations. Thus, in exemplary embodiments, a candidate egl-28 ortholog polynucleotide can be cloned into a vector; a full-length clone corresponding to the egl-28 ortholog can be assembled; the candidate egl-28 ortholog can be expressed; and a knock-out animal in which the candidate egl-28 ortholog is disrupted can be produced using standard techniques. Such knock-out animals can be tested for a response to a neurotransmitter or a psychostimulant.

The invention also provides prescreening and screening methods aimed at identifying an agent that modulates a response to a neurotransmitter or a psychostimulant. A prescreening method of the invention entails contacting a test agent with a polypeptide encoded by a member of the egl-28 gene family or a polynucleotide encoding the polypeptide; and detecting specific binding of the test agent to the polypeptide or polynucleotide. Generally, the member of the egl-28 gene family includes a polypeptide including an amino acid sequence that has at least about 50% identity to the C. elegans egl-28 amino acid sequence (SEQ ID NO:3) over a comparison window of at least 17 contiguous amino acids. Prescreening can be conveniently carried out in vitro. In particular embodiments, the method additionally includes recording any test agent that specifically binds to the polypeptide or polynucleotide in a database of candidate agents that may modulate a response to a neurotransmitter or a psychostimulant.

A screening method of the invention entails contacting a test agent with a cell that expresses a member of the egl-28 gene family; and determining the level of: (i) the polypeptide encoded by the member of the egl-28 gene family; (ii) the RNA transcribed from the member of the egl-28 gene family; or (iii) a neurotransmitter- or a psychostimulant-induced response mediated by the member of the egl-28 gene family. In one embodiment, a neurotransmitter-induced response is determined, and the response includes histamine transport into cells. In another embodiment, a psychostimulant-induced response is determined, and the response includes amphetamine transport into cells. In particular embodiments, the cell used for screening expresses a vertebrate egl-28. Prescreening can be conveniently carried out in vitro. In particular embodiments, the method additionally includes recording any test agent that induces a difference in the level of egl-28 polynucleotide, egl-28 RNA, or neurotransmitter- or psychostimulant-induced response in a database of candidate agents that may modulate a response to a neurotransmitter or a psychostimulant.

The screening method of the invention can, optionally, include selecting a test agent that reduces the level of the egl-28 polypeptide, egl-28 RNA, or neurotransmitter- or psychostimulant-induced response as a candidate inhibitor of a response to the neurotransmitter or the psychostimulant. Similarly, the screening method can include selecting a test agent that increases the level of the polypeptide, RNA, or neurotransmitter- or psychostimulant-induced response as a candidate enhancer of a response to the neurotransmitter or the psychostimulant. In either case, the inhibitor or enhancer, respectively, can, if desired, be combined with a pharmaceutically acceptable carrier.

### BRIEF DESCRIPTION OF THE DRAWINGS

FIG. 1. eg814 mutant animals are strongly and specifically resistant to inhibition of growth and pharyngeal pumping by amphetamine. Worms plated on 5 mM amph at the first larval stage were photographed after three days (A), indicating that while all N2 worms are still in larval stages, many eg814 worms have reached adulthood and begun to lay eggs. This observation is quantified after 5 days in panel B, which indicates that nearly 100% of mutant animals are gravid adults, whereas no wild-type animals are adults. Panel C compares the number of pharyngeal contractions in 15 seconds observed in adult animals plated for 20 hours with (light bars) and without (dark bars) 5 mM amphetamine. Animals were also treated for 90 minutes in solution of 0, 5 and 10 mM amph (dark, light and white bars respectively) and then plated onto agar plates with food for measurement of rate of pumping (D). This resistance to the inhibition of pumping is specific for amphetamine as eg814 mutants are not resistant to the inhibitory effects of muscimol. As shown in panel E, N2 and eg814 animals plated directly onto agar plates containing 0, 0.1 and 1 mM muscimol (light, dark and white bars respectively) exhibited similar inhibition of pumping after both 20 and 45 minutes. *** student's t-test, p<0.001.

FIG. 2. Transformation of eg814 mutants with a small genomic fragment containing a single open-reading frame restores sensitivity to amphetamine. Genetic mapping of the mutation imparting an egg-laying phenotype in eg814 was accomplished by outcrossing of eg814 mutants to a mapping strain CB4856 and analysis of restriction fragment length polymorphisms. We identified a 348.6 kilobase region in the middle of chromosome II which was linked to the egg-laying defect (A). Cosmids within this region were injected and one was found to rescue egg-laying in transformed animals. RNA interference of 65 of the open reading frames within this interval was carried out using bacterial strains from the Ahringer library and a single sequence was found to result in a severe Egl-d phenotype with 100% penetrance. A 4.2 kb genomic fragment containing this open reading frame (W07A12.7) was injected into eg814 worms and shown to rescue the egg-laying and amphetamine resistance phenotypes. As shown in panel B, eg814 animals hold significantly more eggs than N2 worms, this phenotype was reversed in lines transformed with a genomic fragment of eg814 (+) and a GFP marker (egl-28(eg814);[egl-28(+); P_{H2O}::GFP]). When these extrachromosomal arrays were lost (−), the strains reverted to an Egl-d phenotype (B). Panel C shows similarly transformed lines rescued for responsiveness to acute effects of amphetamine on pumping (light bars 0 mM, dark bars 20 mM). The partial resistance shown in the animals which have lost the transgenic array (−) in panel C suggests either some residual transgene below GFP detection levels or some chimeric expression. ** student's t-test, p<0.005.

FIG. 3. Sequence analysis identifies egl-28 mutations in a twelve transmembrane domain protein related to nose resistant to fluoxetine (nrf) mutants. We sequenced exons from the two extant alleles of egl-28 and found guanine to adenosine transitions resulting in glutamic acid to lysine missense mutations in both eg814 and n570 (boxed, A). egl-28 encodes a transmembrane protein which is predicted (see methods) to contain 11 or 12 transmembrane spanning segments (bold underline, panel A). No signal sequence at the N-terminus is apparent and the C-terminus ends in a candidate type II PDZ binding motif. Together this indicates that both N— and C-termini are intracellular and supports the model of an even number of transmembrane domains; additionally, based on Argos trransmembrane predictions (B), we suggest that twelve is the likely number of membrane spanning regions. *, candidate N-linked glycosylation site in the longest extracellular loop. (C) Phylogenetic tree of a small number of the large family of NRF-6 related proteins (Macvector, Oxford Molecular Group). T. tengcongensis (NP_{—}621747.1) is a distantly related receptor from bacteria which serves as outgroup to root the tree. D. melanogaster (NP_{—}608323) is the invertebrate protein most closely related to NRF-6 and EGL-28.

FIG. 4. egl-28 is expressed in head muscle, intestine and a few neurons. Transcriptional fusions of the 1.8 kb regulatory region of egl-28 driving expression of GFP (A, N2;Ex[P_{egl-28}::GFP]) resulted in strong labeling of anterior most head muscle cells in all quadrants (B). A translational fusion was also constructed with a genomic fragment containing 1.8 kb of egl-28 regulatory region and egl-28 cDNA fused to GFP(N2;Ex[P_{egl-28}::egl-28::GFP;ofm-1::GFP]). Animals injected with this construct showed head muscle and intestine expression and strong labeling of RMED and RMEV neuron cell bodies (C). Bright labeling is also seen in coelomocytes (c) due to presence of an ofm-1::GFP co-injection marker.

FIG. 5. Neuronal expression of egl-28 cDNA restores egg-laying and acute amphetamine effects. We analyzed the tissue specific expression effects of egl-28 using regulatory regions from genes encoding egl-28 (egl-28(eg814);Ex[P_{egl-28}::egl-28(+);ofm-1::GFP]), elt-2 (egl-28(eg814);Ex [Pₑₗₜ₋₂::egl-28(+);ofm-1::GFP]), and unc-119 (egl-28(eg814);Ex[P_{unc-119}::egl-28(+);ofm-1::GFP]). The number of eggs held in utero 40 hours after L4 stage were counted by bleaching single animals in individual wells (A). egl-28 cDNA expression driven by both the egl-28 and unc-119 promoters restored wild-type egg-laying. These transgenic lines were also assayed for responsiveness to the acute effects of amphetamine on pumping (B, light bars 0 mM, dark bars 20 mM). Both egl-28 and unc-119 promoters restored responsiveness to nearly wild-type levels. No significant rescue of either egg-laying defects or amphetamine responsiveness was detected with the elt-2 promoter construct. Four independent transformed lines for each genotype were analyzed for rescue of the Egl-d phenotype *** student's t-test, p<0.001.

### DETAILED DESCRIPTION

The present invention provides a method of modulating a response to a neurotransmitter or a psychostimulant drug based on modulating a member of the egl-28 gene family. In particular embodiments, the response of interest is inhibited by employing an inhibitor of the egl-28.

Definitions

Terms used in the claims and specification are defined as set forth below unless otherwise specified.

As used herein, “the egl-28 gene family” refers to the family of genes including all orthologs of egl-28. “Orthologs” are genes that are descended from a common ancestral gene and that share the same function. The terms “a member of the egl-28 gene family” and “egl-28” are used interchangeably herein. Both terms are used to refer to genes, as well as to there encoded proteins. Although one skilled in the art will readily appreciate whether the gene or protein is being discussed, based on context alone, the terms “egl-28 polynucleotide” and “egl-28 polypeptide” are also used herein to ensure greater clarity.

“Psychostimulants” include drugs that stimulate the central nervous system, such as, for example, amphetamine, cocaine, methamphetamine, methylphenidate (ritalin), and methylene dioxy-methamphetamine (MDMA).

A “modulator” of a polypeptide is either an inhibitor or an enhancer of an action or function of the polypeptide.

A modulator “acts directly on” a polypeptide when the modulator exerts its action by interacting directly with the polypeptide.

A modulator “acts indirectly on” a polypeptide when the modulator exerts its action by interacting with a molecule other than the polypeptide, which interaction results in modulation of an action or function of the polypeptide.

An “inhibitor” or “antagonist” of a polypeptide is an agent that reduces, by any mechanism, any action or function of the polypeptide, as compared to that observed in the absence (or presence of a smaller amount) of the agent. An inhibitor of a polypeptide can affect: (1) the expression, mRNA stability, protein trafficking, modification (e.g., phosphorylation), or degradation of a polypeptide, or (2) one or more of the normal actions or functions of the polypeptide.

An “enhancer” or “activator” is an agent that increases, by any mechanism, any polypeptide action or function, as compared to that observed in the absence (or presence of a smaller amount) of the agent. An enhancer of a polypeptide can affect: (1) the expression, mRNA stability, protein trafficking, modification (e.g., phosphorylation), or degradation of a polypeptide, or (2) one or more of the normal actions or functions of the polypeptide.

As used with respect to polypeptides or polynucleotides, the term “isolated” refers to a polypeptide or polynucleotide that has been separated from at least one other component that is typically present with the polypeptide or polynucleotide. Thus, a naturally occurring polypeptide is isolated if it has been purified away from at least one other component that occurs naturally with the polypeptide or polynucleotide. A recombinant polypeptide or polynucleotide is isolated if it has been purified away from at least one other component present when the polypeptide or polynucleotide is produced.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N. Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2, 3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

As used with reference to a polypeptide, the term “full-length” refers to a polypeptide having the same length as the mature wild-type polypeptide. As used with reference to a polynucleotide (e.g., a clone), the term “full-length” refers to a polynucleotide that includes the complete coding sequence for an encoded polypeptide.

“Oligonucleotides” are typically relatively short nucleic acid molecules, generally (although not necessarily) shorter than 200 nucleotides, more particularly, shorter than 100 nucleotides, most particularly, shorter than 50 nucleotides. Typically, oligonucleotides are single-stranded DNA or RNA molecules.

A “subsequence” of an amino acid or nucleotide sequence is a portion of a larger sequence.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Residues in two or more polypeptides are said to “correspond” if they are either homologous (i.e., occupying similar positions in either primary, secondary, or tertiary structure) or analogous (i.e., having the same or similar functional capacities). As is well known in the art, homologous residues can be determined by aligning the polypeptide sequences for maximum correspondence as described above.

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized.

The term “amino acid sequence variant” refers to a polypeptide having an amino acid sequence that differs from a wild-type amino acid sequence by the addition, deletion, or substitution of an amino acid.

The term “conservative amino acid substitution” is used herein to refer to the replacement of an amino acid with a functionally equivalent amino acid. Functionally equivalent amino acids are generally similar in size and/or character (e.g., charge or hydrophobicity) to the amino acids they replace. Amino acids of similar character can be grouped as follows:

(1) hydrophobic: His, Trp, Tyr, Phe, Met, Leu, lie, Val, Ala;

(2) neutral hydrophobic: Cys, Ser, Thr;

(3) polar: Ser, Thr, Asn, Gln;

(4) acidic/negatively charged: Asp, Glu;

(5) charged: Asp, Glu, Arg, Lys, His;

(6) basic/positively charged: Arg, Lys, His;

(7) basic: Asn, Gln, His, Lys, Arg;

(8) residues that influence chain orientation: Gly, Pro; and

(9) aromatic: Trp, Tyr, Phe, His.

The following table shows exemplary and preferred conservative amino acid substitutions.

| Original | Exemplary | Preferred Conservative |
|---|---|---|
| Residue | Conservative Substitution | Substitution |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Asn |
| Ile | Leu, Val, Met, Ala, Phe | Leu |
| Leu | Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala | Leu |

As used with reference to a polypeptide or polypeptide, the term “derivative” includes amino acid sequence variants as well as any other molecule that differs from a wild-type amino acid sequence by the addition, deletion, or substitution of one or more chemical groups. “Derivatives” retain at least one biological or immunological property of a wild-type polypeptide or polypeptide fragment, such as, for example, the biological property of specific binding to a receptor and the immunological property of specific binding to an antibody.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778).

The term “antiserum” refers to a polyclonal antibody typically raised by immunizing an animal with an immunogen and collecting serum containing polyclonal antibodies. The serum may be subjected to one or more purification steps, including affinity purification, to produce the antiserum.

The phrases “an effective amount” and “an amount sufficient to” refer to amounts of a biologically active agent to produce an intended biological activity.

A “signal sequence” is an amino acid sequence that directs the secretion of a polypeptide fused to the signal sequence. As used in recombinant expression, the polypeptide is secreted from a cell expressing the polypeptide into the culture medium for ease of purification.

An “epitope tag” is an amino acid sequence that defines an epitope for an antibody. Epitope tags can be engineered into polypeptides or peptides of interest to facilitate purification or detection. Exemplary epitope tags include the green fluorescent protein (GFP), hemagglutinin, and FLAG epitope tags, which are used in the studies described in Example 1.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

The term “vector” is used herein to describe a DNA construct containing a polynucleotide. Such a vector can be propagated stably or transiently in a host cell. The vector can, for example, be a plasmid, a viral vector, or simply a potential genomic insert. Once introduced into a suitable host, the vector may replicate and function independently of the host genome, or may, in some instances, integrate into the host genome.

As used herein, the term “operably linked” refers to a functional linkage between a control sequence (typically a promoter) and the linked sequence. For example, a promoter is operably linked to a sequence if the promoter can initiate transcription of the linked sequence.

“Expression vector” refers to a DNA construct containing a polynucleotide that is operably linked to a control sequence capable of effecting the expression of the polynucleotide in a suitable host. Exemplary control sequences include a promoter to effect transcription, an optional operator sequence to control transcription, a sequence encoding suitable mRNA ribosome binding sites, and sequences that control termination of transcription and translation.

The term “host cell” refers to a cell capable of maintaining a vector either transiently or stably. Host cells of the invention include, but are not limited to, bacterial cells, yeast cells, insect cells, plant cells and mammalian cells. Other host cells known in the art, or which become known, are also suitable for use in the invention.

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

The term “primer” refers to an oligonucleotide that is capable of hybridizing (also termed “annealing”) with a polynucleotide and serving as an initiation site for nucleotide (RNA or DNA) polymerization.

A “test agent” is any agent that can be screened in the prescreening or screening assays of the invention. The test agent can be any suitable composition, including a small molecule, peptide, or polypeptides.

Methods of Modulating a Response to a Neurotransmitter or a Psychostimulant

The invention provides methods of modulating a response to a neurotransmitter or a psychostimulant.

A. Method Of Inhibiting a Response to a Neurotransmitter or a Psychostimulant

1. In General

Inhibition of egl-28 can reduce or block responses to neurotransmitters, such as histamine, and psychotimulants, such as amphetamine. The inhibition of histamine-induced responses finds application, for example, in the modulation of wakefulness, alertness, and appetite, as well as in diseases in which histamine is implicated, such as schizophrenia. The inhibition of psychostimulant-induced responses finds application, for example, in the treatment of abuse of psychostimulant drugs, as well as in the management of adverse effects of psychostimulants that are used therapeutically. Accordingly, the invention provides a method of inhibiting a response to a neurotransmitter or a psychostimulant, wherein the method entails contacting cells expressing a member of the egl-28 gene family, with an effective amount of an inhibitor of that member of the egl-28 gene family, in the presence the neurotransmitter or the psychostimulant, respectively.

In one embodiment the method is carried out to inhibit a response to neurotransmitters such as histamine, dopamine, serotonin, and norepinephrine, as well as trace amines, such as tyramine and octopamine, and neuropeptides.

In another embodiment, the method is carried out to inhibit a response to a psychostimulant, such as amphetamine, cocaine, methamphetamine, methylphenidate (ritalin), and methylene dioxy-methamphetamine (MDMA), and the like.

Any cell that expresses a member of the egl-28 gene family can be employed in the method. The method generally employs animal cells, typically cells from vertebrates, preferably from birds or mammals, more preferably from animals having research or commercial value or value as pets, such as mice, rats, guinea pigs, rabbits, cats, dogs, chickens, pigs, sheep, goats, cows, horses, as well as monkeys and other primates. Human cells can also be employed. The cell can be, although is not necessarily, one that expresses egl-28 endogenously. Accordingly, cells expressing a heterologous egl-28 from any of the organisms noted above can also be employed in the methods of the invention.

In particular embodiments, the member of the egl-28 gene family is a polypeptide including an amino acid sequence that has at least about 50% identity to egl-28 over a comparison window of at least 17 contiguous amino acids. In variations of this embodiment, the percent identity is at least about: 55, 60, 65, 70, 80, 90, 95, 96, 97, 98, 99, or 100 percent, although lower percent identities are also possible (e.g., at least about: 10, 20, 30, 35, 40, or 45 percent). Alternatively, or in addition, the member of the egl-28 gene family can have at least about 20% identity to the full-length egl-28 amino acid sequence. In variations of this embodiment, the percent identity is at least about: 25, 30, 35, 40, 50, 60, 70, 80, 90, 95, 96, 97, 98, 99, or 100 percent, although lower percent identities are also possible (e.g., at least about: 5, 10, or 15 percent). In a specific example of this embodiment, the amino acid sequence includes the amino acid sequence of C. elegans egl-28 (SEQ ID NO:3).

Any kind of egl-28 inhibitor that is tolerated by the cells can be employed in the method of the invention. Thus, the inhibitor can be a polypeptide (such as, e.g., an anti-egl-28 antibody), a polynucleotide (e.g., an inhibitory RNA or a polynucleotide that encodes an inhibitory polypeptide), or a small molecule. In particular embodiments, when the inhibitor is a polynucleotide encoding an inhibitory polypeptide, the polynucleotide is introduced into the cells, where the encoded polypeptide is expressed in an amount sufficient to inhibit egl-28.

The cells are generally contacted with inhibitor under physiological conditions before or during a period of contact with a neurotransmitter and/or psychostimulant. The duration of contact with the inhibitor can vary, depending on the particular application of the method. The duration of contact can range from minutes to days or longer. For research applications, the inhibitor is typically contacted with cells for, e.g., about 30 mins.; or about 1, about 3, about 6, or about 12 hours; or about 1, about 2, about 5, about 10, or about 15 days.

Contact of the inhibitor with cells can be achieved directly, i.e., by administering a composition containing the inhibitor to the cells, or indirectly, e.g., by administering a composition containing a polynucleotide encoding an inhibitor polypeptide to the cells. In the latter embodiment, this administration results in the introduction of the polynucleotide into one or more cells and the subsequent expression of the polypeptide in an amount sufficient to inhibit egl-28 in the cells.

This method can be carried out in vitro, i.e., in cells or tissues that are not part of an organism, or in vivo, in cells that are part of an organism. In one embodiment, cells are contacted in vitro in with an effective amount of an inhibitor (or a polynucleotide encoding the inhibitor).

Alternatively, cells can be contacted in vivo with a inhibitor by administering a composition containing the inhibitor (or a polynucleotide encoding the inhibitor) directly to a suitable subject. In particular embodiments, for example, the subject is undergoing treatment for psychostimulant abuse or is being treated using a psychostimulant.

Inhibition of egl-28 can be achieved by any available means, e.g., modulation of: (1) the expression, mRNA stability, protein trafficking, modification (e.g., phosphorylation), or degradation of egl-28, or (2) one or more of the normal functions of egl-28, such as neurotransmitter and/or psychostimulant binding and/or transport.

In certain embodiments, the egl-28 inhibitor can be, e.g., a peptide or a small molecule identified through a screening assay of the invention, which are described below.

In other embodiments, egl-28 inhibition is achieved by reducing the level of egl-28 polypeptides in the cells or inhibiting egl-28 function by various means that entail introducing polynucleotide inhibitors into cells. egl-28 levels can be reduced using, e.g., antisense, catalytic RNA/DNA, RNA interference (RNAᵢ), or “knock-out” techniques. egl-28 expression/function can be inhibited using intrabodies.

a. Antisense Methods

egl-28 gene expression can be reduced or entirely blocked by the use of antisense molecules. An “antisense sequence or antisense polynucleotide” is a polynucleotide that is complementary to the egl-28 coding mRNA sequence or a subsequence thereof. Binding of the antisense molecule to the egl-28 mRNA interferes with normal translation of the egl-28 polypeptide.

Thus, in particular embodiments, the invention provides antisense molecules useful for inhibiting egl-28. Suitable antisense molecules include oligonucleotides and oligonucleotide analogs that are hybridizable with egl-28 mRNA. The oligonucleotides and oligonucleotide analogs are able to inhibit the function of the RNA, either its translation into protein, its translocation into the cytoplasm, or any other activity necessary to its overall biological function. The failure of the mRNA to perform all or part of its normal functions results in a partial or complete inhibition of expression of egl-28 polypeptides.

“Oligonucleotides useful in the antisense methods of the invention include polynucleotides formed from naturally-occurring bases and/or cyclofuranosyl groups joined by native phosphodiester bonds. The term “oligonucleotide” encompasses moieties that function similarly to oligonucleotides, but that have non-naturally occurring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short-chain alkyl or cycloalkyl structures. In accordance with other preferred embodiments, the phosphodiester bonds are substituted with structures that are, at once, substantially non-ionic and non-chiral, or with structures which are chiral and enantiomerically specific. Persons of ordinary skill in the art will be able to select other linkages for use in the practice of the invention.

In an exemplary embodiment, the internucleotide phosphodiester linkage is replaced with a peptide linkage. Such peptide polynucleotides tend to show improved stability, penetrate the cell more easily, and show enhanced affinity for their target. Methods of making peptide polynucleotides are known to those of skill in the art (see, e.g., U.S. Pat. Nos: 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides useful in the antisense methods of the invention may also include one or more modified base forms. Thus, purines and pyrimidines other than those normally found in nature may be employed. Similarly, the furanosyl portions of the nucleotide subunits may also be modified, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are: OH, SH, SCH₃, F, OCH₃, OCN, O(CH₂)[n]NH₂ or O(CH₂)[n]CH₃, where n is from 1 to about 10, and other substituents having similar properties.

All such analogs can be used in the antisense methods of the invention so long as the analogs function effectively to hybridize with egl-28 nRNA and inhibit the function of that RNA.

Antisense oligonucleotides in accordance with this invention preferably comprise from about 3 to about 50 subunits (i.e., bases in unmodified polynucleotides). It is more preferred that such oligonucleotides and analogs comprise from about 8 to about 25 subunits and still more preferred to have from about 12 to about 20 subunits. The oligonucleotides used in accordance with this invention can be conveniently and routinely made through the well-known technique of solid phase synthesis. Equipment for such synthesis is sold by several vendors (e.g. Applied Biosystems).

Antisense oligonucleotides of the invention can be synthesized, formulated, and administered to cells, tissues, or organisms in accordance with standard practice. General considerations with respect to administration and dose are discussed below. Formulations containing at least one component that facilitates entry of a polynucleotide into a cell are discussed below with respect to compositions containing polynucleotides encoding egl-28 polynucleotides. Those of skill in the art will readily appreciate that this discussion is equally applicable to antisense oligonucleotides, catalytic RNAs and DNAs, and double-stranded RNAs used in RNAi. Similarly, those of skill in the art understand that antisense oligonucleotides can be introduced into host cells as described below for egl-28 polynucleotides.

b. Catalytic RNAs and DNAs

(1) Ribozymes

In another approach, egl-28 expression can be inhibited by the use of ribozymes. As used herein, “ribozymes” include RNA molecules that contain antisense sequences for specific recognition, and an RNA-cleaving enzymatic activity. The catalytic strand cleaves a specific site in a target (egl-28) RNA, preferably at greater than stoichiometric concentration. The ribozymes of the invention typically consist of RNA, but such ribozymes may also be composed of polynucleotide molecules comprising chimeric polynucleotide sequences (such as DNA/RNA sequences) and/or polynucleotide analogs (e.g., phosphorothioates).

Accordingly, one aspect of the present invention includes ribozymes have the ability to inhibit egl-28 expression. Such ribozymes may, e.g., be in the form of a “hammerhead” (for example, as described by Forster and Symons (1987) Cell 48: 211-220; Haseloff and Gerlach (1988) Nature 328: 596-600; Walbot and Bruening (1988) Nature 334: 196; Haseloff and Gerlach (1988) Nature 334: 585); Rossi et al. (1991) Pharmac. Ther. 50: 245-254) or a “hairpin” (see, e.g., U.S. Pat. No. 5,254,678 and Hampel et al., European Patent Publication No. 0 360 257, published Mar. 26, 1990; Hampel et al. (1990) Nucl. Acids Res. 18: 299-304), and have the ability to specifically target and cleave and egl-28 polynucleotides.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO:1). Suitable egl-28 recognition or target sequences for hairpin ribozymes can be readily determined from the egl-28 sequence.

The sequence requirement at the cleavage site for the hammerhead ribozyme is any RNA sequence consisting of NUX (where N is any of G, U, C, or A and X represents C, U, or A). Accordingly, the same target within the hairpin leader sequence, GUC, is useful for the hammerhead ribozyme. The additional nucleotides of the hammerhead ribozyme or hairpin ribozyme are determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an 8-base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. Preferred ribozymes of the invention hybridize efficiently to target sequences at physiological temperatures, making them particularly well suited for use in vivo.

Ribozymes, as well as DNA encoding such ribozymes, and other suitable polynucleotide molecules can be chemically synthesized using methods well known in the art for the synthesis of polynucleotide molecules. Alternatively, Promega, Madison, Wis., USA, provides a series of protocols suitable for the production of RNA molecules such as ribozymes. The ribozymes also can be prepared from a DNA molecule or other polynucleotide molecule (which, upon transcription, yields an RNA molecule) operably linked to an RNA polymerase promoter, e.g., the promoter for T7 RNA polymerase or SP6 RNA polymerase (e.g., a vector that provides an intiation site and template for transcription). Accordingly, also provided by this invention are polynucleotide molecules, e.g., DNA or cDNA, coding for the ribozymes of this invention. When the vector also contains an RNA polymerase promoter operably linked to the polynucleotide molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the corresponding phosphothio analog for use in liposome delivery systems. This modification also renders the ribozyme resistant to endonuclease activity.

Ribozymes, or polynucleotides encoding them (e.g., DNA vectors) can be formulated, and administered to cells, tissues, or organisms in accordance with standard practice. General considerations with respect to administration and dose are discussed below. Formulations containing at least one component that facilitates entry of a polynucleotide into a cell are discussed below with respect to compositions containing polynucleotides encoding egl-28 polynucleotides. Those of skill in the art will readily appreciate that this discussion is equally applicable to antisense oligonucleotides, catalytic RNAs and DNAs, and double-stranded RNAs used in RNAi. Similarly, those of skill in the art understand that antisense oligonucleotides can be introduced into host cells as described below for egl-28 polynucleotides.

When a vector containing an encoded ribozyme linked to a promoter for RNA transcription, the RNA can be produced in the host cell when the host cell is grown under suitable conditions favoring transcription of the vector. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W.H. Freeman and Company, New York.

To produce ribozymes in vivo utilizing such vectors, the nucleotide sequence endoding the ribozyme is preferably operably linked to a strong promoter such as the lac, SV40 late, SV40 early, or lambda promoters.

(2) Catalytic DNA

In a manner analogous to ribozymes, DNA molecules are also capable of catalytic (e.g. nuclease) activity. For example, highly catalytic species have been developed by directed evolution and selection. Beginning with a population of 10¹⁴ DNAs containing 50 random nucleotides, successive rounds of selective amplification enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is composed of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹min⁻¹ under multiple turnover conditions, exceeding that of any other known polynucleotide enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to egl-28 mRNA and can be used in essentially the same manner as described above for egl-28 ribozymes.

c. RNAi Methods

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. This approach is based on the observation that injection of antisense or sense RNA strands into C. elegans cells resulted in gene-specific inactivation (Guo and Kempheus (1995) Cell 81: 611-620). While gene inactivation by the antisense strand was expected, gene silencing by the sense strand was unexpected. Surprisingly, it was determined that the gene-specific inactivation was actually due to trace amounts of contaminating dsRNA (Fire et al. (1998) Nature 391: 806-811).

Since then, this mode of post-transcriptional gene silencing has been demonstrated in a wide variety of organisms: plants, flies, trypanosomes, planaria, hydra, zebrafish, and mice (Zamore et al. (2000) Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expression, which do not allow for gene silencing during various stages of the developmental process (Gura (2000) Nature 404:804-808).

dsRNA can be formulated, and administered to cells, tissues, or organisms in accordance with standard practice. General considerations with respect to administration and dose are discussed below, as are formulations containing at least one component that facilitates entry of a polynucleotide into a cell (discussed below with respect to compositions containing polynucleotides encoding egl-28 polynucleotides). Those of skill in the art will readily appreciate that dsRNA can be introduced into host cells as described below for egl-28 polynucleotides. Additionally, dsRNA can be synthesized using one or more vectors designed to transcribe the two complementary RNA strands that hybridize to form the dsRNA (see the discussion of this approach with respect to ribozymes, above). These may be introduced into host cells using any of the techniques described herein or known in the art for this purpose.

After introduction into cells, it has been shown that dsRNA is cleaved by a nuclease into 21-23-nucleotide fragments. These fragments, in turn, target the homologous region of their corresponding mRNA, hybridize, and result in a double-stranded substrate for a nuclease that degrades it into fragments of the same size (Hammond et al. (2000) Nature 404:293-298; Zamore et al. (2000) Cell 101:25-33).

d. “Knock-Out” Methods

In another approach, egl-28 can be inhibited simply by “knocking out” the egl-28 gene. Typically, this is accomplished by disrupting the egl-28 gene, the promoter regulating the gene or sequences between the promoter and the gene. Such disruption can be specifically directed to egl-28 by homologous recombination where a “knockout construct” contains flanking sequences complementary to the domain to which the construct is targeted. Insertion of the knockout construct (e.g., into the egl-28 gene) results in disruption of that gene. The phrases “disruption of the gene” and “gene disruption” refer to insertion of a nucleic acid sequence into one region of the native DNA sequence (usually one or more exons) and/or the promoter region of a gene so as to reduce or prevent expression of that gene in the cell, as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The genomic DNA sequence to be used in producing the knockout construct is digested with a particular restriction enzyme selected to cut at a location(s) such that a new DNA sequence encoding, e.g., a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable; however, typically it is an antibiotic resistance gene or other gene whose expression or presence in the genome can easily be detected. The marker gene is usually operably linked to its own promoter or to another strong promoter from any source that will be active, or can easily be activated, in the cell into which it is introducied; however, the marker gene need not be linked to its own promoter as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to, neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement).

The resulting knockout constructs can be delivered to cells in vivo using gene therapy delivery vehicles (e.g., retroviruses, liposomes, lipids, dendrimers, etc.). Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

Although embryonic stem (ES) cells can be employed to produce knockout animals, ES cells are not required. In various embodiments, knockout animals can be produced using methods of somatic cell nuclear transfer. In preferred embodiments using such an approach, a somatic cell is obtained from the species in which the egl-28 gene is to be knocked out. The cell is transfected with a construct that introduces a disruption in the egl-28 gene (e.g., via homologous recombination). Cells harboring a knocked out egl-28 gene are selected, e.g., by selecting for expression of a marker encoded by a marker gene used to disrupt the native gene. The nucleus of cells harboring the knockout is then placed in an unfertilized enucleated egg (e.g., eggs from which the natural nuclei have been removed by microsurgery). Once the transfer is complete, the recipient eggs contain a complete set of genes, just as they would if they had been fertilized by sperm. The eggs are then cultured for a period before being implanted into a host mammal (of the same species that provided the egg) where they are carried to term, culminating in the birth of a transgenic animal comprising a nucleic acid construct containing one or more disrupted egl-28 gene.

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Somatic cell nuclear transfer simplifies transgenic procedures by employing a differentiated cell source that can be clonally propagated. This eliminates the need to maintain the cells in an undifferentiated state, thus, genetic modifications, both random integration and gene targeting, are more easily accomplished. Also, by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

e. Intrabodies

In still another embodiment, egl-28 expression/activity can be inhibited by introducing a nucleic acid construct that expresses an intrabody into the target cells. An intrabody is an intracellular antibody, in this case, capable of recognizing and binding to an egl-28 polypeptide. The intrabody is expressed by an “antibody cassette” containing: (1) a sufficient number of nucleotides encoding the portion of an antibody capable of binding to the target (egl-28 polypeptide) operably linked to (2) a promoter that will permit expression of the antibody in the cell(s) of interest. The construct encoding the intrabody is delivered to the cell where the antibody is expressed intracellularly and binds to the target egl-28, thereby disrupting the target from its normal action.

In a preferred embodiment, the “intrabody gene” of the antibody cassette includes a cDNA encoding heavy chain variable (V_{H}) and light chain variable (V_{L}) domains of an antibody which can be connected at the DNA level by an appropriate oligonucleotide linker, which on translation, forms a single peptide (referred to as a single chain variable fragment, “sFv”) capable of binding to a target such as an egl-28 protein. The intrabody gene preferably does not encode an operable secretory sequence, and thus the expressed antibody remains within the cell.

Anti-egl-28 antibodies suitable for use/expression as intrabodies in the methods of this invention can be readily produced by a variety of methods, some of which are describe below with respect to anti-elg28 antibodies of the invention. Such methods include, but are not limited to, traditional methods of raising polyclonal antibodies, which can be modified to form single chain antibodies, or screening of, e.g., phage display libraries to select for antibodies showing high specificity and/or avidity for egl-28.

The antibody cassette is delivered to the cell by any means suitable for introducing polynucleotides into cells. A preferred delivery system is described in U.S. Pat. No. 6,004,940. Methods of making and using intrabodies are described in detail in U.S. Pat. Nos. 6,072,036, 6,004,940, and 5,965,371.

2. Administration

For in vitro applications, cells are contacted with an inhibitor of the invention simply by adding the inhibitor or the polynucleotide encoding the inhibitor directly to the medium of cultured cells or directly to tissues.

Methods for in vivo administration do not differ from known methods for administering drugs or therapeutic polypeptides, peptides, or polynucleotides encoding them. Suitable routes of administration include, for example, topical, intravenous, intraperitoneal, intracerebral, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

3. Dose

The dose of inhibitor is sufficient to reduce or block one or more egl-28-mediated actions without significant toxicity. For in vivo applications, the dose of inhibitor depends, for example, upon the therapeutic objectives, the route of administration, and the condition of the subject. Accordingly, it is necessary for the clinician to titer the dosage and modify the route of administration as required to obtain the optimal therapeutic or other beneficial effect. Generally, the clinician begins with a low dose and increases the dosage until the desired therapeutic effect is achieved. Starting doses for a given inhibitor can be extrapolated from in vitro data.

B. Method of Enhancing a Response to a Neurotransmitter or a Psychostimulant

Enhancement of egl-28 can increase responses to neurotransmitters, such as histamine, and psychotimulants, such as amphetamine. The enhancement of such responses has a variety of uses in the research setting, for example in further elucidating the signaling pathways in which egl-28 participates. In addition, enhancement of egl-28 can be used therapeutically, for example, in treating conditions that are amenable to treatment using psychostimulants, such as amphetamine (e.g., attention deficit disorder, fatigue [i.e, to produce increased wakefulness or alertness], and weight loss, etc.). Accordingly, the invention provides a method of enhancing a response to a neurotransmitter or a psychostimulant, wherein the method entails contacting cells expressing a member of the egl-28 gene family, with an effective amount of an enhancer of that member of the egl-28 gene family. In particular embodiments, the cells are contacted with the enhancer in the presence the neurotransmitter or the psychostimulant, respectively.

In one embodiment the method is carried out to enhance a response to a neurotransmitter, such as histamine, dopamine, serotonin, and norepinephrine, as well as a trace amine, such as tyramine and octopamine, or a neuropeptide.

In another embodiment, the method is carried out to inhibit a response to a psychostimulant, such as amphetamine, cocaine, methamphetamine, methylphenidate (ritalin), and methylene dioxy-methamphetamine (MDMA), and the like.

Any cell that expresses a member of the egl-28 gene family can be employed in the method of enhancing a response to a neurotransmitter or a psychostimulant. Suitable cells are as described above with respect to inhibiting a response to a neurotransmitter or a psychostimulant.

In particular embodiments, the member of the egl-28 gene family is a polypeptide including an amino acid sequence that has at least about 50% identity to egl-28 over a comparison window of at least 17 contiguous amino acids. In variations of this embodiment, the percent identity is at least about: 55, 60, 65, 70, 80, 90, 95, 96, 97, 98, 99, or 100 percent, although lower percent identities are also possible (e.g., at least about: 10, 20, 30, 35, 40, or 45 percent). Alternatively, or in addition, the member of the egl-28 gene family can have at least about 20% identity to the full-length egl-28 amino acid sequence. In variations of this embodiment, the percent identity is at least about: 25, 30, 35, 40, 50, 60, 70, 80, 90, 95, 96, 97, 98, 99, or 100 percent, although lower percent identities are also possible (e.g., at least about: 5, 10, or 15 percent). In a specific example of this embodiment, the amino acid sequence includes the amino acid sequence of C. elegans egl-28 (SEQ ID NO:3).

Any kind of egl-28 enhancer that is tolerated by the cells can be employed in the method of the invention. Thus, the inhibitor can be a polypeptide (such as, e.g., a transcriptional activator or an anti-egl-28 antibody), a polynucleotide, or a small molecule. In particular embodiments, when the enhancer is a polynucleotide encoding an enhancing polypeptide, the polynucleotide is introduced into the cells, where the encoded polypeptide is expressed in an amount sufficient to enhance egl-28.

Enhancement of egl-28 can be achieved by any available means, e.g., modulation of: (1) the expression, mRNA stability, protein trafficking, modification (e.g., phosphorylation), or degradation of egl-28, or (2) one or more of the normal functions of egl-28, such as neurotransmitter and/or psychostimulant binding and/or transport.

In one embodiment, egl-28 is enhanced by increasing the level of egl-28 polypeptides in the cells. Egl-28 enhancers suitable for use in the invention can be, e.g., a peptide or a small molecule identified through a screening assay of the invention, which are described below.

The cells are generally contacted with enhancer under physiological conditions. In certain embodiments, the cells are contacted with the enhancer before or during a period of contact with a neurotransmitter and/or psychostimulant. The duration of contact with the enhancer can vary, depending on the particular application of the method. The duration of contact can range from minutes to days or longer. For research applications, the inhibitor is typically contacted with cells for, e.g., about 30 mins.; or about 1, about 3, about 6, or about 12 hours; or about 1, about 2, about 5, about 10, or about 15 days.

Contact of the enhancr with cells can be achieved directly, i.e., by administering a composition containing the inhibitor to the cells, or indirectly, e.g., by administering a composition containing a polynucleotide encoding an inhibitor polypeptide to the cells. In the latter embodiment, this administration results in the introduction of the polynucleotide into one or more cells and the subsequent expression of the polypeptide in an amount sufficient to enhancer egl-28 in the cells.

This method can be carried out in vitro, i.e., in cells or tissues that are not part of an organism, or in vivo, in cells that are part of an organism. In one embodiment, cells are contacted in vitro in with an effective amount of an egl-28 enhancer (or a polynucleotide encoding the egl-28 enhancer).

Alternatively, cells can be contacted in vivo with a egl-28 enhancer by administering a composition containing the egl-28 enhancer (or a polynucleotide encoding the egl-28 enhancer) directly to a subject.

The general considerations affecting administration and dose of egl-28 enhancers do not differ from those set forth above with respect to the method of inhibiting a response to a neurotransmitter or a psychostimulant.

egl-28 Polypeptides

A. Types of egl-28 Polypeptides

The invention also provides egl-28 polypeptides. A egl-28 polypeptide of the invention includes a egl-28 amino acid sequence, i.e., an amino acid sequence that has at least about 50% identity to the C. elegans egl-28 amino acid sequence (SEQ ID NO:3) over a comparison window of at least 17 contiguous amino acids, provided said polypeptide is not nfr-6 (Genbank accession no. NM_{—}063279) or ndg-4 (Genbank accession no. NM_{—}065506). In variations of this embodiment, the percent identity is 10, 20, 30, 35, 40, 45, 55, 60, 65, 70, 80, 90, 95, 96, 97, 98, 99, or 100 percent. Percent identity can, for example, be determined by a sequence alignment performed using BLASTP with default parameters set to measure the selected percent identity. egl-28 amino acid SEQ ID NO:3 is the amino acid sequence of a C. elegans egl-28 polypeptide described in detail in Example 1. The nucleic acid and (single-letter code) amino acid sequences of this polypeptide are given below.

### C. elegans egl-28 Nucleic Acid (cDNA) Sequence

atgtcatcatcgccacacactcacactctaataatcatggaacgactcagcgaagtgcgattgtactcatcaccaatgacatcagtact caactcaaatctatcttcttgcttaacaaatgcatttattcctgatactgtacgaatggccagtagtccacttttggcatggatgacacttgt cgtcatcggaacacttgctccagtaagcgtcttttcctgcttgagcctcaagcgatcagtcaaagaattgctcactgaaagatcctcga aactcgatgtattggatatcttccgattcgttgcaatcctttgggttatgcttaaccatactggaagtgaaggaagaattgatattttggatc gactaccatctgctgatgcattcaaaagtgcaatgcatgatcatccaatttttggagctctcatgggaaactctgctcttggagttgaaat tttccttgtactttctggacttttggcagctagatcgtggcttcgtaaagccgatgagccatttttccaacattggaaatcatttattgctcgt agactactccgcttggctccatccatgttcatttttgtctacatcgctgctggtccaatcatgaatgctcttctcccacgatactcctcttca atggtctccgcttgtggtttttggggtattctttcccatgtaacattcacttctaattggcaatccacaccaacttgcatgggatatctttggt atttgggactcgacatgcaactttacatggttgctccaatcttcttgaatcttcttcacaagtttccaaaacgtggaatggctctcactatta ccactataattgcctctatggtcatccgtgcgggttactgtaccgcctatggaacttgcaaccaaagtgatgtcgatattccattcatttca tatccagggcaagacgcagagacattgaagagtatttatgctggactttgggacatgtactcaagaccatataccaagtgtggtccatt ccttatcggtcttcttctcggatacatcacagtttccagcaaatacatcatggtttcgactacatccaagacactcttccgttccagtctcat tgtcgcaatcgccacaatctacgcaattctcccagaatattggaacccaaatgctggaaatactctctataatacagtttacacggcagt attccgatctgtattcgctatggctatttctggaatgattgctgctctgtatttcagacaagaataccgtccaacaaatccgatcttcgcca tgcttgccaagctcacctacaacgcgtatctccttcatatgccagttgtctatattttcaattggctcccattccttcaagctgccacttcac caattcatcttctactagttcttccatttgttgcaattctatcattcattgctgctctcatcttttatcttttcattgaggccccaattgggcatttg acatctcaatatgccacacgactgggcctgtag SEQ ID NO:2

### C. elegans egl-28 Amino Acid Sequence

| | |
|---|---|
| MSSSPHTHTLIIMERLSEVRLYSSPMTSVLNSNLSSCLT | SEQ ID NO:3 |
| NAFIPDTVRMASSPLLAWMTLVVIGTLAPVSVFSCLSLK | |
| RSVKELLTERSSKLDVLDIFRFVAILWVMLNHTGSEGRI | |
| DILDRLPSADAFKSAMHDHPIFGALMGNSALGVEIFLVL | |
| SGLLAARSWLRKADEPFFQHWKSFIARRLLRLAPSMFIF | |
| VYIAAGPIMNALLPRYSSSMVSACGFWGILSHVTFTSNW | |
| QSTPTCMGYLWYLGLDMQLYMVAPIFLNLLHKFPKRGMA | |
| LTITTIIASMVIRAGYCTAYGTCNQSDVDIPFISYPGQD | |
| AETLKSIYAGLWDMYSRPYTKCGPFLIGLLLGYITVSSK | |
| YIMVSTTSKTLFRSSLIVAIATIYAILPEYWNPNAGNTL | |
| YNTVYTAVFRSVFAMAISGMIAALYFRQEYRPTNPIFAM | |
| LAKLTYNAYLLHMPVVYIFNWLPFLQAATSPIHLLLVLP | |
| FVAILSFIAALIFYLFIEAPIGHLTSQYATRLGL | |

The invention also encompasses polypeptides wherein the percent identities noted above are found over a comparison window of at least 20, 30, 40, 50, 60, 70, 80, 90, 100, 200, 300, 400, 500, or more contiguous amino acid, up to, and including, the full-length of one or both polypeptides. In a particular embodiments, an egl-28 polypeptide of the invention includes an amino acid sequence has at least about 20% identity to the full-length the C. elegans egl-28 amino acid sequence (SEQ ID NO:3). In a variation of this embodiment, the amino acid sequence comprises the amino acid sequence of C. elegans egl-28 (SEQ ID NO:3).

The egl-28 amino acid sequence can be derived from any egl-28-like polypeptide from any organism. egl-28 amino acid sequences useful in the invention are generally those derived from vertebrates, preferably from birds or mammals, more preferably from animals having research or commercial value or value as pets, such as mice, rats, guinea pigs, rabbits, cats, dogs, chickens, pigs, sheep, goats, cows, horses, as well as monkeys and other primates. In particularly preferred embodiments, the egl-28 amino acid sequence is derived from a human egl-28 polypeptide. The nucleic acid and (single-letter code) amino acid sequences of the human polypeptide are given below.

### Human egl-28 Nucleic Acid (cDNA) Sequence

| | |
|---|---|
| atgcgtatatgttatgaatgccaaaatgaaagaacattg | SEQ ID NO:4 |
| tggcgatgtgtttcccaggatggggctgactacagtgtg | |
| ggcgtgtgtgtccctgattcttgtgctgaagaggatgtg | |
| actctgatgtctcggctggatactttaagattcagaaat | |
| acttcatttttggccccttccctctttctttttacaata | |
| aattcttcctccttgtctggtgggagtgtgaccagatgt | |
| gctgctggaaagatccccctggacacatttgctgccgta | |
| tgtctgttcatcaccttgctgggtctcatcctccctccg | |
| gctggaacagtctgcgtggcagctagggaatgggggtca | |
| gcctgcaggacatcgcgggaacacggggaacctctggcc | |
| acttacgggagtctgccactgagcgaggcggagagcaat | |
| gaacaaagaagcagaatcccacggacacactgccgggca | |
| catctcctcctgtcagcagcctccagcagaggaaaaagg | |
| tttctaggagccgtggctcatgctctggagtgcttttct | |
| tggcagaagaatgtgccagccatctggactacaaaggca | |
| ccaggtggcacctgctctgcactgaatggcattcgtgtc | |
| ttgagtcttctttggatcatctcgggacacaccagtcag | |
| atgactgcatggctgtctttgggatggaaagatggaggg | |
| cacgaaaggccactggtcatgtctgggccatcagtggga | |
| atcggagacaccagagaagccacgagtggttggttaagt | |
| gcaagttcgtttttaaagatgcatcagaattcagacaaa | |
| ggaataacccccaaaggcatactcagatactttctcagt | |
| cacctggtaaggttgcagcctcttcacctgtattcaatg | |
| tgcttgttggttggactgttctctcttgttccctgggga | |
| cctgtctgggaaatgcccaaattccactgggataactgc | |
| cggcaagcatggtggacgaatctgctgttgctaaataac | |
| tttgtgtcggtcaagaatgcgtgcaatggctggacctgg | |
| taccttgccaatgacttccagttccacctcaccacacca | |
| gtgattatcttcatccatgtaaagagtacacagatcctc | |
| atcctccttggggccatgctgttcttggcatctttcaca | |
| gccactgctctgatcaccttggcatataaacttcctgtc | |
| gtggctccatcagaaaccaggacttcccggggagggctg | |
| ctgaatgccaggctgttcaccctgtgccctttggttcat | |
| ggaaaaagtgggtatgaaacttttggtctggatgggaaa | |
| gctgattgccttcttgcttccaaacttctgaacctttca | |
| acctgcactggaaatgaacaagtgtgccctaaatgtacc | |
| tttgggcttgctgattattccaatggacatctcagggat | |
| ttggattccctttgccatgtccagatcaaacataacatt | |
| ttggcttatttccttgtatttttcagtgaagaggcgatt | |
| gtattgtatttcgtggagtactacacaaagccctactgc | |
| cgatttgggccagttcttgtgggcctctttctgagcatt | |
| tacatgcaccaaaaccaccaggaaaacattctcagaacc | |
| aagctgcagctctctaccaagccctccaccggaccctgt | |
| gggcggcggctgtgggctgagtcctctttgcgtgccacg | |
| gaggatatggaggtatggaagcggctccaggctttgctg | |
| tcgggttcacaccctgttcctttaaaggtgacaaatcga | |
| acacacaggagagccaagcagataaaaggcttcaatgga | |
| aaagaatcttctccaggtctggtgaaccgtgtgctttct | |
| tgggacatctggagtttcctgtccagcatcagttatgct | |
| cgctacttggtgcatccgattctgatcatcctttacaat | |
| ggccttcaggaaacacttattcaccacactgacaccaac | |
| atgttctatcttttctctggacaccgtgtgctgaccttc | |
| gtcactgggctggccctgacgctgttcattgagaaacca | |
| tgtcaggaactgaagcagcacctgctgggccatgaatgt | |
| tctggttaa | |

### Human egl-28 Amino Acid Sequence

| | |
|---|---|
| MRICYECQNERTLWRCVSQDGADYSVGVCVPDSCAEEDV | SEQ ID NO:5 |
| TLMSRLDTLRFRNTSFLAPSLFLFTINSSSLSGGSVTRC | |
| AAGKIPLDTFAAVCLFITLLGLILPPAGTVCVAAREWGS | |
| ACRTSREHGEPLATYGSLPLSEAESNEQRSRIPRTHCRA | |
| HLLLSAASSRGKRFLGAVAHALECFSWQKNVPAIWTTKA | |
| PGGTCSALNGIRVLSLLWIISGHTSQMTAWLSLGWKDGG | |
| HERPLVMSGPSVGIGDTREATSGWLSASSFLKMHQNSDK | |
| GITPKGILRYFLSHLVRLQPLHLYSMCLLVGLFSLVPWG | |
| PVWEMPKFHWDNCRQAWWTNLLLLNNFVSVKNACNGWTW | |
| YLANDFQFHLTTPVIIFIHVKSTQILILLGAMLFLASFT | |
| ATALITLAYKLPVVAPSETRTSRGGLLNARLFTLCPLVH | |
| GKSGYETFGLDGKADCLLASKLLNLSTCTGNEQVCPKCT | |
| FGLADYSNGHLRDLDSLCHVQIKHNILAYFLVFFSEEAI | |
| VLYFVEYYTKPYCRFGPVLVGLFLSIYMHQNHQENILRT | |
| KLQLSTKPSTGPCGRRLWAESSLRATEDMEVWKRLQALL | |
| SGSHPVPLKVTNRTHRRAKQIKGFNGKESSPGLVNRVLS | |
| WDIWSFLSSISYARYLVHPILIILYNGLQETLIHHTDTN | |
| MFYLFSGHRVLTFVTGLALTLFIEKPCQELKQHLLGHEC | |
| SG | |

In preferred embodiments, egl-28 polypeptides of the invention include and amino acid sequence that defines a multi-pass transmembrane protein.

In various embodiments, the amino acid sequence includes an amino acid subsequence of at least about 10, 25, 50, 75, 100, 150, 200, 250, 300, 350, 400, 450, 500 or more amino acids, of any of the above-described egl-28 polypeptides. Subsequences can be limited to no more than about 25, 50, 75, 100, 150, 200, 250, 300, 350, 400, 450, or 500 amino acids.

The egl-28 amino acid sequence can be a wild-type amino acid sequence or an amino acid sequence variant of the corresponding region of a wild-type polypeptide. Preferred egl-28 polypeptides generally include a wild-type egl-28 amino acid sequence or a egl-28 amino acid sequence containing conservative amino acid substitutions, as defined above.

In addition to the amino acid sequences described above, egl-28 polypeptides of the invention can include other amino acid sequences, including those from heterologous proteins. Accordingly, the invention encompasses fusion polypeptides in which the above-discussed amino acid sequence is fused, at either or both ends, to amino acid sequence(s) from one or more heterologous proteins. Examples of additional amino acid sequences often incorporated into proteins of interest include a signal sequence, which facilitates recombinant production of the protein, and an epitope tag, which can be used for immunological detection or affinity purification.

Polypeptides of the invention can be otherwise modified to produce derivatives that retain at least one characteristic or function of egl-28, e.g., ability to form a a multi-pass transmembrane protein and/or modulation of a neurotransmitter- or psychostimulant-induced effect. In preferred embodiments, the modified polypeptides have an activity that is about 0.1 to about 0.01-fold that of the unmodified forms. In more preferred embodiments, the modified polypeptides have an activity that is about 0.1 to about 1-fold that of the unmodified polypeptides. In even more preferred embodiments, the modified polypeptides have an activity that is greater than the unmodified polypeptides.

Those of skill in the art recognize that a variety of techniques are available for constructing so-called “peptide mimetics” with the same or similar desired biological activity as the corresponding peptide compound, but with more favorable activity than the peptide with respect to, e.g., solubility, stability, and susceptibility to hydrolysis and proteolysis. See, for example, Morgan, et al., Ann. Rep. Med. Chem., 24:243-252 (1989). Accordingly, the egl-28 polypeptides of the invention include peptide mimetics that are, for example, modified at the N-terminal amino group, the C-terminal carboxyl group, and/or at one or more of the amido linkages in the peptide to a non-amido linkage.

B. Production of egl-28 Polypeptides

1. Synthetic Techniques

egl-28 polypeptides according to the invention can be synthesized using methods known in the art, such as for example exclusive solid phase synthesis, partial solid phase synthesis, fragment condensation, and classical solution synthesis. See, e.g., Merrifield, J. Am. Chem. Soc., 85:2149 (1963). Solid phase techniques are preferred. On solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories, Richmond, Calif., and the preparation of the hydroxymethyl resin is described by Bodonszky, et al., Chem. Ind. (London), 38:1597 (1966). The benzhydrylamine (BHA) resin has been described by Pietta and Marshall, Chem. Commn., 650 (1970) and is commercially available from Beckman Instruments, Inc., Palo Alto, Calif., in the hydrochloride form. Automated peptide synthesizers are commercially available, as are services that make peptides to order.

Thus, the polypeptides of the invention can be prepared by coupling an alpha-amino protected amino acid to the chloromethylated resin with the aid of, for example, cesium bicarbonate catalyst, according to the method described by Gisin, Helv. Chim. Acta., 56:1467 (1973). After the initial coupling, the alpha-amino protecting group is removed by a choice of reagents including trifluoroacetic acid (TFA) or hydrochloric acid (HCl) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, acetyl), aromatic urethane type protecting groups (e.g. benzyloxycarboyl (Cbz) and substituted Cbz), aliphatic urethane protecting groups (e.g., t-butyloxycarbonyl (Boc), isopropyloxycarbonyl, cyclohexyloxycarbonyl), and alkyl type protecting groups (e.g., benzyl, triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride, dimethyl formamide (DMF) mixtures.

After the desired amino acid sequence has been completed, the desired peptide is decoupled from the resin support by treatment with a reagent such as trifluoroacetic acid or hydrogen fluoride (HF), which not only cleaves the peptide from the resin, but also cleaves all remaining side chain protecting groups. When the chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well known in the art and further described by John Morrow Stewart and Janis Dillaha Young, Solid Phase Peptide Syntheses (2nd Ed., Pierce Chemical Company, 1984).

2. Recombinant Techniques

egl-28 polypeptides can also produced using recombinant techniques. Precursor egl-28 genes or gene sequences can be cloned, for instance, based on homology to the egl-28 polypeptides described herein. With a precursor egl-28 gene in hand, a nucleic acid molecule encoding a desired egl-28 polypeptide can be generated by any of a variety of mutagenesis techniques. See, e.g., Sambrook, J., Fritsch, E. F., and Maniatis, T. (1989) in Molecular Cloning: A Laboratory Manual, Second Edition, Cold Spring Harbor, N.Y. Examples include site-specific mutagenesis (Kunkel et al., (1991) Methods Enzymol., 204:125-139; Carter, P., et al., (1986) Nucl. Acids Res. 10:6487), cassette mutagenesis (Wells, J. A., et al., (1985) Gene 34:315), and restriction selection mutagenesis (Wells, J. A., et al., (1986) Philos. Trans. R. Soc., London Ser. A, 317:415).

In a preferred embodiment of the invention, the sequence of a egl-28 coding region is used as a guide to design a synthetic nucleic acid molecule encoding the egl-28 polypeptide that can be incorporated into a vector of the present invention. Methods for constructing synthetic genes are well-known to those of skill in the art. See, e.g., Dennis, M. S., Carter, P. and Lazarus, R. A. (1993) Proteins: Struct. Funct. Genet., 15:312-321. Expression and purification methods are described below in connection with the nucleic acids, vectors and host cells of the invention.

C. Uses of egl-28 Polypeptides

The egl-28 polypeptides of the invention are useful in a variety of research and therapeutic applications. The discovery of egl-28 polypeptides that mediate neurotransmitter- and psychostimulant-induced responses will facilitate studies aimed at elucidating the series of molecular events underlying these phenomena. In other research applications, the egl-28 polypeptides of the invention can be used in screening assays (see below) to identify additional molecules that inhibit or enhance such responses. In addition, egl-28 polypeptides can be used as standards in immunoassays to detect the presence of egl-28 polypeptides in a biological sample.

egl-28 polypeptides can also be used therapeutically to treat conditions that can be ameliorated by modulating neurotransmitter and/or psychostimulant-induced responses that rely on egl-28. For example, egl-28 polypeptides could be employed in treating conditions that are amenable to treatment using psychostimulants, such as amphetamine (e.g., attention deficit disorder, weight loss, etc.). Pharmaceutical compositions containing the polypeptides of the invention are described in greater detail below.

Polynucleotides, Vectors, and Host Cells

The invention also provides a polynucleotide encoding a polypeptide of the invention, a vector including this polynucleotide, and a host cell including the vector.

A. Polynucleotides

Polynucleotides of the invention include a portion that encodes an egl-28 polypeptide. As noted above, the encoded egl-28 amino acid sequence can be a wild-type sequence or a variant sequence. Where the egl-28 amino acid sequence is a wild-type sequence, the nucleotide sequence encoding this sequence can be a wild-type nucleotide sequence or one containing “silent” mutations that do not alter the amino acid sequence due to the degeneracy of the genetic code. For example, if the polynucleotide is intended for use in expressing the encoded polypeptide, silent mutations can be introduced by standard mutagenesis techniques to optimize codons to those preferred by the host cell.

In some applications, it is advantageous to stabilize the polynucleotides described herein or to produce polynucleotides that are modified to better adapt them for particular applications. To this end, the polynucleotides of the invention can contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH2-NH—O—CH2, CH2-N(CH3)-O—CH2 (known as the methylene(methylimino) or MMI backbone) and CH2-O—N(CH3)-CH2, CH2-N(CH3)-N(CH3)-CH2, and O—N(CH3)-CH2-CH backbones (where phosphodiester is O—P—O—CH2). Also preferred are polynucleotides having morpholino backbone structures. Summerton, J. E. and Weller, D. D., U.S. Pat. No. 5,034,506. Other preferred embodiments use a protein-nucleic acid or peptide-nucleic acid (PNA) backbone, wherein the phosphodiester backbone of the polynucleotide is replaced with a polyamide backbone, the bases being bound directly or indirectly to the aza nitrogen atoms of the polyamide backbone. P. E. Nielsen, M. Egholm, R. H. Berg, O. Buchardt, Science 1991, 254, 1497. Polynucleotides of the invention can contain alkyl and halogen-substituted sugar moieties and/or can have sugar mimetics such as cyclobutyls in place of the pentofuranosyl group. In other preferred embodiments, the polynucleotides can include at least one modified base form or “universal base” such as inosine. Polynucleotides can, if desired, include an RNA cleaving group, a cholesteryl group, a reporter group, an intercalator, a group for improving the pharmacokinetic properties of the polynucleotide, and/or a group for improving the pharmacodynamic properties of the polynucleotide.

Those of skill in the art understand that polynucleotides complementary to the coding strand of polynucleotides of the invention can be employed to inhibit expression of the polypeptides of the invention, which may be of interest for research or therapeutic purposes. Accordingly, the nucleic acids of the invention include such “antisense polynucleotides,” and the phrase “polynucleotide encoding a polypeptide of the invention” is intended to include such antisense molecules.

B. Vectors

A polynucleotide of the present invention can be incorporated into a vector for propagation and/or expression in a host cell. Such vectors typically contain a replication sequence capable of effecting replication of the vector in a suitable host cell (i.e., an origin of replication) as well as sequences encoding a selectable marker, such as an antibiotic resistance gene. Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art.

If the vector is intended for expression of a polypeptide, the vector includes one or more control sequences capable of effecting and/or enhancing the expression of an operably linked polypeptide coding sequence. Control sequences that are suitable for expression in prokaryotes, for example, include a promoter sequence, an operator sequence, and a ribosome binding site. Control sequences for expression in eukaryotic cells include a promoter, an enhancer, and a transcription termination sequence (i.e., a polyadenylation signal).

An expression vector according to the invention can also include other sequences, such as, for example, nucleic acid sequences encoding a signal sequence or an amplifiable gene. A signal sequence can direct the secretion of a polypeptide fused thereto from a cell expressing the protein. In the expression vector, nucleic acid encoding a signal sequence is linked to a polypeptide coding sequence so as to preserve the reading frame of the polypeptide coding sequence. The inclusion in a vector of a gene complementing an auxotrophic deficiency in the chosen host cell allows for the selection of host cells transformed with the vector.

A vector of the present invention is produced by linking desired elements by ligation at convenient restriction sites. If such sites do not exist, suitable sites can be introduced by standard mutagenesis (e.g., site-directed or cassette mutagenesis) or synthetic oligonucleotide adaptors or linkers can be used in accordance with conventional practice.

Viral vectors are of particular interest for use in delivering polynucleotides of the invention to a cell or organism, followed by expression of the encoded protein, i.e., “gene therapy” when performed to ameliorate a pathological condition. For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088. Adenoviral vectors suitable for use in the invention are also commercially available. For example, the Adeno-X™ Tet-Off™ gene expression system, sold by Clontech, provides an efficient means of introducing inducible heterologous genes into most mammalian cells.

Adeno-associated virus (AAV)-based vectors used to transduce cells with target nucleic acids, e.g., in the in vitro production of nucleic acids and peptides, and in vivo and ex vivo gene therapy procedures are describe, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

C. Host Cells

The present invention also provides a host cell containing a vector of this invention. A wide variety of host cells are available for propagation and/or expression of vectors. Examples include prokaryotic cells (such as E. coli and strains of Bacillus, Pseudomonas, and other bacteria), yeast or other fungal cells (including S. cerevesiae and P. pastoris), insect cells, plant cells, and phage, as well as higher eukaryotic cells (such as human embryonic kidney cells and other mammalian cells). Host cells according to the invention include cells in culture and cells present in live organisms, such as transgenic plants or animals or cells into which a gene therapy vector has been introduced.

A vector of the present invention is introduced into a host cell by any convenient method, which will vary depending on the vector-host system employed. Generally, a vector is introduced into a host cell by transformation (also known as “transfection”) or infection with a virus (e.g., phage) bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by infection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, and protoplast fusion also are acceptable for use in the invention.

Other procedures include the use of polynucleotides linked to an inactive adenovirus (Cotton et al. (1990) Proc. Natl. Acad. Sci., USA, 89 :6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor-specific ligands, liposomes entrapping the polynucleotide molecules, spheroplast fusion whereby E coli containing the polynucleotide molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus.

Recombinant Production Methods

Host cells transformed with expression vectors can be used to express the polypeptides encoded by the polynucleotides of the invention. Expression entails culturing the host cells under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well-known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium or a cell lysate by any method capable of separating the polypeptide from one or more components of the host cell or culture medium. Typically, the polypeptide is separated from host cell and/or culture medium components that would interfere with the intended use of the polypeptide. As a first step, the culture medium or cell lysate is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an epitope tag or other affinity domain, purification typically includes the use of an affinity column containing the cognate binding partner. For instance, polypeptides fused with green fluorescent protein, hemagglutinin, or FLAG epitope tags or with hexahistidine or similar metal affinity tags can be purified by fractionation on an affinity column.

Compositions

A. In General

For research and therapeutic applications, the egl-28 polypeptides of the invention, or polynucleotides encoding them, can be specifically formulated for administration to cells, tissues, or organisms. These compositions are generally formulated to deliver egl-28 polypeptides to a target site in an amount sufficient to modulate (inhibit or enhance) a response to a neurotransmitter or a psychostimulant. In certain embodiments, the compositions can also include a neurotransmitter and/or a psychostimulant, generally in an amount sufficient to elicit a response at the target site.

B. Compositions Containing egl-28 Polypeptides

The invention provides compositions, including pharmaceutical compositions, containing a polypeptide of the invention. The compositions optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Preferred embodiments include sustained-release pharmaceutical compositions. An exemplary sustained-release composition has a semipermeable matrix of a solid hydrophobic polymer to which the polypeptide is attached or in which the polypeptide is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

Exemplary sustained release compositions include polypeptides attached, typically via ε-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” variant retains the desired function(s).

In another embodiment, a sustained-release composition includes a liposomally entrapped polypeptide. Liposomes are small vesicles composed of various types of lipids, phospholipids, and/or surfactants. These components are typically arranged in a bilayer formation, similar to the lipid arrangement of biological membranes. Liposomes containing polypeptides are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions can also include a polypeptide adsorbed onto a membrane, such as a silastic membrane, which can be implanted, as described in International Publication No. WO 91/04014.

Pharmaceutical compositions of the invention can be stored in any standard form, including, e.g., an aqueous solution or a lyophilized cake. Such compositions are typically sterile when administered to recipients. Sterilization of an aqueous solution is readily accomplished by filtration through a sterile filtration membrane. If the composition is stored in lyophilized form, the composition can be filtered before or after lyophilization and reconstitution.

C. Compositions Containing Polynucleotides Encoding egl-28 Polypeptides

The invention provides compositions, including pharmaceutical compositions, containing a polynucleotide encoding the polypeptide of the invention. Such compositions optionally include other components, as for example, a storage solution, such as a suitable buffer, e.g., a physiological buffer. In a preferred embodiment, the composition is a pharmaceutical composition and the other component is a pharmaceutically acceptable carrier as described above.

In preferred embodiments, compositions containing polynucleotides of the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with the polynucleotides of the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC). Polynucleotides can also be entrapped in liposomes, as described above for polypeptides.

In another embodiment, polynucleotides are complexed to dendrimers, which can be used to transfect cells. Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides of the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides. In a preferred embodiment, therapeutic compositions of the invention include polynucleotides combined with lipids, as described above.

Compositions containing polynucleotides can be stored in any standard form, including, e.g., an aqueous solution or a lyophilized cake. Such compositions are typically sterile when administered to cells or recipients. Sterilization of an aqueous solution is readily accomplished by filtration through a sterile filtration membrane. If the composition is stored in lyophilized form, the composition can be filtered before or after lyophilization and reconstitution.

Anti-egl-28 Antibodies

The invention includes an antibody and an antiserum that specifically recognizes a egl-28 polypeptide of the invention. Thus, the invention encompasses polyclonal and monoclonal anti-egl-28 antibodies. Polyclonal antibodies are raised by injecting (e.g. subcutaneous or intramuscular injection) antigenic polypeptides into a suitable non-human mammal (e.g., a mouse or a rabbit). Any suitable egl-28 polypeptide described herein, or fragment thereof, may be employed to raise anti-egl-28 antibodies. Generally, the polypeptide used as the immungen should induce production of high titers of antibody with relatively high affinity for egl-28.

If desired, the immunizing polypeptide may be conjugated to a carrier protein by conjugation using techniques that are well known in the art. Commonly used carriers include keyhole limpet hemocyanin (KLH), thyroglobulin, bovine serum albumin (BSA), and tetanus toxoid. The conjugate is then used to immunize the animal.

The antibodies are then obtained from blood samples taken from the animal. The techniques used to produce polyclonal antibodies are extensively described in the literature (see, e.g., Methods of Enzymology, “Production of Antisera With Small Doses of Immunogen: Multiple Intradermal Injections,” Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal, as well as monoclonal, antibodies see, for example, Coligan, et al. (1991) Unit 9, Current Protocols in Immunology, Wiley Interscience).

For many applications, monoclonal anti-egl-28 antibodies are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mAbs can be accomplished using relatively routine screening techniques (such as the enzyme-linked immunosorbent assay, or “ELISA”) to determine the elementary reaction pattern of the mAb of interest.

It is also possible to evaluate a mAb to determine whether it has the same specificity as a mAb described herein without undue experimentation by determining whether the mAb being tested prevents the described mAb from binding a target polypeptide. If the mAb being tested competes with the mAb described herein, it is likely that the two monoclonal antibodies bind to the same or a closely related epitope. Still another way to determine whether a mAb has the specificity of a mAb described herein is to preincubate the mAb described herein with an antigen with which it is normally reactive, and determine if the mAb being tested is inhibited in its ability to bind the antigen. Such inhibition indicates that the mAb being tested has the same, or a closely related, epitopic specificity as the mAb described herein.

As used herein, the term “antibody” encompasses antigen-binding antibody fragments, e.g., single chain antibodies (scFv or others), which can be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIlI fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20-fold -1,000,000-fold are obtained for a single round of affinity selection. By infecting bacteria with the eluted phage, however, more phage can be grown and subjected to another round of selection. In this way, an enrichment of 1000-fold in one round can become 1,000,000-fold in two rounds of selection (McCafferty et al. (1990) Nature, 348: 552-554). Thus, even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural VH and VL repertoires present in human peripheral blood lymphocytes are isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

As those of skill in the art readily appreciate, antibodies can be prepared by any of a number of commercial services (e.g., Berkeley antibody laboratories, Bethyl Laboratories, Anawa, Eurogenetec, etc.).

Methods of Identifying egl-28 Orthologs

The invention also provides a method of identifying an egl-28 ortholog. In one embodiment, the method entails determining whether a candidate egl-28 ortholog polynucleotide comprises a nucleotide sequence that is substantially similar to an egl-28 nucleotide sequence. In another embodiment, the method entails determining whether a candidate egl-28 ortholog polypeptide comprises an amino acid sequence that is substantially similar to an egl-28 amino acid sequence.

Sequence comparisons to determine whether two sequences represent orthologs are carried out using any suitable method known to those skilled in the art. For instance, phylogenetic analyses of amino acid sequences can be performed using MacVector, as described in Example 1. In certain embodiments, substantially similar amino acid sequences share at least about 50% identity over a comparison window of at least 17 contiguous amino acids. Alternatively, or in addition, the amino acid sequences can have at least about 20% identity over the entire length of one or both sequences.

In certain embodiments, the method is directed to identifying one or more vertebrate orthologs. For example, the method can be employed to identify orthologs from birds or mammals, more preferably from animals having research or commercial value or value as pets, such as mice, rats, guinea pigs, rabbits, cats, dogs, chickens, pigs, sheep, goats, cows, horses, as well as monkeys and other primates and, in particular, humans.

The invention also included a variety of manipulations of candidate egl-28 orthologs that can be carried out on candidate egl-28 orthologs identified as discussed above or in some other manner. Thus, for example, in one embodiment, the invention includes cloning a candidate egl-28 ortholog polynucleotide into a vector, such as are described above. In variation of the embodiment, a full-length clone corresponding to the egl-28 ortholog is assembled using standard recombinant techniques. Where the vector is an expression vector, the candidate egl-28 ortholog can be produced recombinantly, e.g., as described above. In an additional embodiment, the invention includes producing a knock-out animal in which the candidate egl-28 ortholog is disrupted, as described above. In a variation of this embodiment, the knock-out animal is tested for a response to a neurotransmitter or a psychostimulant. Generally, the test will represent an established animal model for studying a response to the particular neurotransmitter (e.g., histamine) or psychostimulant (e.g., amphetamine). Exemplary tests for amphetamine responses in C. elegans are described in Example 1.

Methods of Screening for Agents that Modulate a Response to a Neurotransmitter or a Psychostimulant

The role of egl-28 polypeptides in mediating neurotransmitter and psychostimulant-induced responses makes the egl-28 an attractive target for agents that modulate such responses. Accordingly, the invention provides prescreening and screening methods aimed at identifying agents that either inhibit or enhance neurotransmitter or psychostimulant responses. Such methods are useful, for example, to screen for agents that produce the desirable affects of psychostimulants, such as amphetamines, without the undesirable side effects. The prescreening/screening methods of the invention are generally, although not necessarily, carried out in vitro. Accordingly, screening assays are generally carried out, for example, using purified or partially purified components (egl-28 polypeptides or polynucleotides, etc.), in cell lysates, in cultured cells, or in a biological sample.

A. Prescreening Based on Binding to egl-28 Polypeptides or Polynucleotides

The prescreening methods are based on screening test agents for specific binding, either to an egl-28 polypeptide or polynucleotide.

In one embodiment, therefore, a prescreening method of the invention entails contacting a test agent with an egl-28 polypeptide, followed by detection of specific binding of the test agent to the egl-28 polypeptide. Suitable egl-28 polypeptides include an amino acid sequence that has at least about 50% identity to egl-28 (SEQ ID NO:3) over a comparison window of at least 17 contiguous amino acids. Thus, egl-28 polypeptides described herein, as well as fragments thereof, can be employed in the screening method.

In an alternative embodiment, the test agent can be contacted with a polynucleotide encoding the egl-28 polypeptide to screen for agents that affect egl-28 expression, followed by detection of specific binding of the test agent to the egl-28 polynucleotide.

Such prescreening is generally most conveniently accomplished with a simple in vitro binding assay. Means of assaying for specific binding of a test agent to a polypeptide or polynucleotide are well known to those of skill in the art. In preferred binding assays, the egl-28 polypeptide of polynucleotide is immobilized and exposed to a test agent (which can be labeled), or alternatively, the test agent(s) are immobilized and exposed to the egl-28 polypeptide or polynucleotide (which can be labeled). The immobilized moiety is then washed to remove any unbound material and the bound test agent or bound egl-28 polypeptide or polynucleotide is then detected. To prescreen large numbers of test agents, high throughput assays are generally preferred. Various prescreening formats are discussed in greater detail below.

B. Screening Based on Levels of egl-28 Polypeptide or RNA

Test agents, including, for example, those identified in a prescreening assay of the invention can also be screened to determine whether the test agent affects the levels of egl-28 polypeptide or RNA. Agents that reduce these levels can potentially inhibit egl-28-mediated responses. Conversely, agents that increase these levels can potentially enhance egl-28-mediated responses.

Accordingly, the invention provides a method of screening for an agent that modulates a response to a neurotransmitter or a psychostimulant in which a test agent is contacted with a cell that expresses a member of the egl-28 gene family. After contact with the test agent, the level of egl-28 polypeptide or RNA is determined in the presence and absence (or presence of a lower amount) of test agent to determine whether the test agent modulated the level.

Cells useful in this screening method include those described above with respect to methods of inhibiting a response to a neurotransmitter or a psychostimulant. Cells that naturally express egl-28 are typically, although not necessarily, employed in this screening method. Preferred cells include vertebrate cells, particularly mammalian cells, and more particularly human cells.

1. Sample Collection and Processing

As noted above, screening assays are generally carried out in vitro, for example, in cell lysates, in cultured cells, or in a biological sample. (e.g., neural tissue) derived from an animal, preferably a mammal, and more preferably from a human. For ease of description, cell cultures and biological samples are referred to as “samples” below.

The sample may be pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

2. Polypeptide-Based Assays

egl-28 polypeptide(s) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one embodiment, the egl-28 polypeptide(s) are detected/quantified in an electrophoretic polypeptide separation (e.g. a 1- or 2-dimensional electrophoresis). Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence of egl-28 polypeptide(s) in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with antibodies that specifically bind the target polypeptide(s). Antibodies that specifically bind to the target polypeptide(s) and may be directly labeled or alternatively may be detected subsequently using labeled antibodies (e.g., labeled sheep anti-mouse antibodies) that specifically bind to a domain of the primary antibody.

In a preferred embodiment, the egl-28 polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Conventional immunoassays often utilize a “capture agent” to specifically bind to and often immobilize the analyte (in this case a egl-28 polypeptide). In preferred embodiments, the capture agent is an antibody.

Immunoassays also typically utilize a labeling agent to specifically bind to and label the binding complex formed by the capture agent and the target polypeptide. The labeling agent may itself be one of the moieties making up the antibody/target polypeptide complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/target polypeptide complex. Other polypeptides capable of specifically binding immunoglobulin constant regions, such as polypeptide A or polypeptide G may also be used as the label agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured target polypeptide is directly measured. In competitive assays, the amount of target polypeptide in the sample is measured indirectly by measuring the amount of an added (exogenous) polypeptide displaced (or competed away) from a capture agent by the target polypeptide present in the sample. In one competitive assay, a known amount of, in this case, labeled egl-28 polypeptide is added to the sample, and the sample is then contacted with a capture agent. The amount of labeled egl-28 polypeptide bound to the antibody is inversely proportional to the concentration of egl-28 polypeptide present in the sample.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies useful in these immunoassays include polyclonal and monoclonal antibodies, which can be produced, for example, as described above.

3. Polynucleotide Based Assays

Changes in egl-28 expression level can be detected by measuring changes in mRNA and/or a polynucleotide derived from the mRNA (e.g., reverse-transcribed cDNA, etc.).

a. Polynucleotide Sample

The polynucleotide sample is, in certain embodiments, isolated from a biological sample according to any of a number of methods well known to those of skill in the art. For example, methods of isolation and purification of polynucleotides are described in detail in by Tijssen ed., (1993) Chapter 3 of Laboratory Techniques in Biochemistry and Molecular Biology: Hybridization With Polynucleotide Probes, Part I. Theory and Polynucleotide Preparation, Elsevier, N.Y. and Tijssen ed.

In a preferred embodiment, “total” RNA is isolated from a given sample using, for example, an acid guanidinium-phenol-chloroform extraction method, and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987)).

Frequently, it is desirable to amplify the polynucleotide sample prior to assaying for expression level. One of skill in the art will appreciate that whatever amplification method is used, if a quantitative result is desired, care must be taken to use a method that maintains or controls for the relative frequencies of the amplified polynucleotides.

Methods of “quantitative” amplification are well known to those of skill in the art. For example, quantitative PCR involves simultaneously co-amplifying a known quantity of a control sequence using the same amplification primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

b. Hybridization-Based Assays

(i) Detection of Gene Transcripts

Methods of detecting and/or quantifying the transcript(s) of one or more egl-28 gene(s) (e.g. mRNA or cDNA made therefrom) using polynucleotide hybridization techniques are known to those of skill in the art (see Sambrook et al. supra). For example, the presence, absence, or quantity of a reverse-transcribed cDNA can be measured by Southern blot. Alternatively, in a Northern blot, mRNA is directly quantitated. In both cases, labeled probes are used to identify and/or quantify the target mRNA.

The probes used herein for detection of the egl-28 polynucleotides can be full-length or less than the full-length of these polynucleotides. Shorter probes are empirically tested for specificity. Preferably polynucleotide probes are 20 bases or longer in length. (See Sambrook et al. for methods of selecting polynucleotide probe sequences for use in polynucleotide hybridization.) Visualization of the hybridized probes allows the qualitative determination of the presence or absence of the egl-28 polynucleotide, and standard methods (such as, e.g., densitometry) can be used to quantify the level of the egl-28 polynucleotide.

(ii) Amplification-Based Assays

In still another embodiment, amplification-based assays can be used to measure egl-28 expression level. In such amplification-based assays, the target polynucleotide sequences act as template(s) in amplification reaction(s) (e.g., Polymerase Chain Reaction (PCR) or reverse-transcription PCR (RT-PCR)). In a quantitative amplification, the amount of amplification product is proportional to the amount of template in the original sample. Detailed protocols for quantitative PCR are provided in Innis et al. (1990) PCR Protocols, A Guide to Methods and Applications, Academic Press, Inc. N.Y.).

Other suitable amplification methods include, but are not limited to, ligase chain reaction (LCR) (see Wu and Wallace (1989) Genomics 4: 560; Landegren et al. (1988) Science 241: 1077; and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

c. Hybridization Formats and Conditions

(i) Array-Based Hybridization Formats

In one embodiment, the screening methods of this invention can be carried out in an array-based hybridization format. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly polynucleotide arrays can be produced according to a wide variety of methods well known to those of skill in the art. For example, in a simple embodiment, “low density” arrays can simply be produced by spotting (e.g. by hand using a pipette) different polynucleotides at different locations on a solid support (e.g. a glass surface, a membrane, etc.). This simple spotting, approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide microarrays. Synthesis of high density arrays is also described in U.S. Pat. Nos. 5,744,305; 5,800,992; and 5,445,934.

In one embodiment, the arrays used in this invention are arrays “probe” polynucleotides. These probes are then hybridized respectively with their “target” polynucleotides (e.g., mRNA derived from a biological sample). The arrays can be hybridized with a single population of sample polynucleotide or can be used with two differentially labeled collections (as with a test sample and a reference sample). Alternatively, the format can be reversed, such that polynucleotides from different samples (i.e., the target polynucleotides) are arrayed and this array is then probed with one or more probes, which can be differentially labeled.

Many methods for immobilizing polynucleotides on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, and the like. In addition, substances that form gels can be used. Such materials include, e.g., proteins (e.g., gelatins), lipopolysaccharides, silicates, agarose, and polyacrylamides.

In preparing the surface, any of a variety of different materials may be employed, particularly as laminates, to provide desirable properties. For example, proteins (e.g., bovine serum albumin) or mixtures of macromolecules (e.g., Denhardt's solution) can be employed to reduce non-specific binding, simplify covalent conjugation, or enhance signal detection. If covalent bonding between a compound and the surface is desired, the surface will usually be polyfunctional or be capable of being polyfunctionalized. Functional groups which may be present on the surface and used for linking polynucleotides can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. For example, polynucleotides can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). In addition, polynucleotides are conveniently modified by introduction of various functional groups that facilitate immobilization (see, e.g., Bischoff (1987) Anal. Biochem., 164: 336-344; Kremsky (1987) Nucl. Acids Res. 15: 2891-2910).

Arrays can be made up of target elements of various sizes, ranging from 1 mm diameter down to 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

(ii) Other Hybridization Formats

A variety of other hybridization formats are known to those skilled in the art and suitable for use in the screening methods of the invention. Hybridization techniques are generally described in Hames and Higgins (1985) Polynucleotide Hybridization, A Practical Approach, IRL Press; Gall and Pardue (1969) Proc. Natl. Acad. Sci. USA 63: 378-383; and John et al. (1969) Nature 223: 582-587. Common hybridization formats include sandwich assays and competition or displacement assays.

The sensitivity of hybridization assays may be enhanced through use of a polynucleotide amplification system that multiplies the target polynucleotide being detected. Examples of such systems include the polymerase chain reaction (PCR) system and the ligase chain reaction (LCR) system. Other methods recently described in the art are the polynucleotide sequence based amplification (NASBAO, Cangene, Mississauga, Ontario) and Q Beta Replicase systems.

(iii) Hybridization Conditions

Polynucleotide hybridization simply involves providing a denatured probe and target polynucleotide under conditions where the probe and its complementary target can form stable hybrid duplexes through complementary base pairing. The polynucleotides that do not form hybrid duplexes are then washed away leaving the hybridized polynucleotides to be detected, typically through detection of an attached detectable label. Polynucleotides are generally denatured by increasing the temperature or decreasing the salt concentration of the buffer containing the polynucleotides, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In general, there is a tradeoff between hybridization specificity (stringency) and signal intensity. In a preferred embodiment, the wash is performed at the highest stringency that produces consistent results and that provides a signal intensity greater than approximately 10% of the background intensity. Hybridization can performed at low stringency to ensure hybridization and then subsequent washes are performed to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be included in the reaction mixture.

Methods of optimizing hybridization conditions are well known to those of skill in the art (see, e.g., Tijssen (1993) Laboratory Techniques in Biochemistry and Molecular Biology, Vol. 24: Hybridization With Polynucleotide Probes, Elsevier, N.Y.). In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

(iv) Labeling and Detection of Polynucleotides

In a preferred embodiment, the hybridized polynucleotides are detected by detecting one or more labels attached to the sample polynucleotides. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oregon, USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label may be added to the target (sample) polynucleotide(s) prior to, or after the hybridization. So-called “direct labels” are detectable labels that are directly attached to or incorporated into polynucleotide probes prior to hybridization. In contrast, so-called “indirect labels” typically bind to the hybrid duplex after hybridization. Often, the indirect label binds to a moiety that is attached to or incorporated into the polynucleotide probe prior to the hybridization. Thus, for example, the polynucleotide probe may be biotinylated before the hybridization. After hybridization, an avidin-conjugated fluorophore will bind the biotin bearing hybrid duplexes providing a label that is easily detected. For a detailed review of methods of labeling polynucleotides and detecting labeled hybridized polynucleotides see Laboratory Techniques in Biochemistry and Molecular Biology, Vol. 24: Hybridization With Polynucleotide Probes, P. Tijssen, ed. Elsevier, N.Y. (1993).

The labels may be incorporated by any of a number of means well known to those of skill in the art. Means of attaching labels to polynucleotides include, for example nick translation or end-labeling.

D. Screening Based on Level of a Neurotransmitter or a Psychostimulant-Induced Response

The invention also provides a screening method based on determining the effect, if any, of a test agent on the level of a neurotransmitter- or a psychostimulant-induced response mediated by a member of the egl-28 gene family. To screen for an effect on egl-28-mediated responses, the method entails contacting the test agent with a cell that expresses a member of the egl-28 gene family. After contact with the test agent, the neurotransmitter- or a psychostimulant-induced response is determined in the presence and absence (or presence of a lower amount) of test agent to determine whether the test agent modulated the response.

Cells useful in this screening method include those described above with respect to methods of inhibiting a response to a neurotransmitter or a psychostimulant. Cells that naturally express egl-28 are typically, although not necessarily, employed in this screening method. Preferred cells include vertebrate cells, particularly mammalian cells, and more particularly human cells.

In one embodiment, the method entails screening for a neurotransmitter-induced response, such as, for example, histamine transport into cells. Histamine transport can be assayed by any convenient standard biochemical method. In another embodiment, the method entails screening for a psychostimulant-induced response, such as, for example, amphetamine transport into cells. Amphetamine transport can be measured using any any convenient standard biochemical method.

Although in vitro methods are generally preferred, e.g., for high throughput screening. In vivo methods, exemplified herein with C. elegans can also be employed. Thus, for example, the effect of test agents on psychostimulant-induced C. elegans growth, pharyngeal contractions (pumping), egg laying, and locomotion can be determined, as described in Example 1. Histamine-induced nose contraction is an example of a neurotransmitter-induced response that may be examined in this system; histamine-induced nose contraction can be measured. In preferred embodiments, these responses can be tested in wild-type animals and in animals lacking functional egl-28 polypeptide (e.g., the eg814 mutant described in Example 1). If a response that is affected in the same manner in wild-type an mutant animals, this would indicate that the effect on the response is mediated by a pathway other than the egl-28 pathway.

E. Test Agent Databases

In a preferred embodiment, generally involving the screening of a large number of test agents, the screening method includes the recordation of any test agent that identified in any of the prescreening/screening methods of the invention in a database of candidate agents that may modulate a response to a neurotransmitter or a psychostimulant.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

Test Agents Identified by Screening

When a test agent is found to reduce the level of egl-28 polypeptide or RNA or to reduce a neurotransmitter- or psychostimulant-induced response, a preferred screening method of the invention further includes selecting the test agent as a candidate inhibitor of a response to the neurotransmitter or the psychostimulant. Conversely, when a test agent is found to increase the level of egl-28 polypeptide or RNA or to increase a neurotransmitter- or psychostimulant-induced response, a preferred screening method of the invention further includes selecting the test agent as a candidate enhancer of a response to the neurotransmitter or the psychostimulant.

In both embodiments, methods of the invention optionally include combining the inhibitor or enhancer with a carrier, preferably pharmaceutically acceptable carrier, such as are described above. Generally, the concentration of inhibitor or enhancer is sufficient to inhibit or enhance, respectively, the relevant neurotransmitter- or psychostimulant-induced response when the composition is contacted with a cell, as described above for the egl-28 -containing compositions and the methods of the invention for modulating neurotransmitter and psychostimulant responses. This concentration will vary, depending on the particular inhibitor/enhancer and specific application for which the composition is intended. As one skilled in the art appreciates the considerations affecting the formulation of a test inhibitor/enhancer with a carrier are generally the same as described above.

### EXAMPLE

The following example is offered to illustrate, but not to limit, the claimed invention.

### Example 1

### egl-28 Encodes a Transmembrane Protein Required for Amphetamine Sensitivity in C. elegans

Summary

Amphetamine is a strong psychostimulant that is widely abused. Amphetamine also has wide ranging therapeutic uses that are limited by its neurotoxic and addictive properties. Some of the neurobehavioral effects of amphetamine are likely to be mediated by inhibition of synaptic transporters responsible for reuptake of the brain neurotransmitters dopamine and serotonin. However, other behavioral and neurotoxic effects of amphetamine have not clearly been linked to altered monoamine reuptake. To identify additional targets of amphetamine, we have used a novel genetic selection in the nematode Caenorhabditis elegans to isolate amphetamine resistant mutants. One of the mutants, egl-28(eg814), exhibits strong resistance to amphetamine inhibition of growth as well as resistance to behavioral effects of the drug. Here we show that egl-28 encodes a multi-pass transmembrane protein related to a class of putative transporters defined by the C. elegans fluoxetine (Prozac) resistant mutants nrf-6 and ndg-4. Members of this family are found in other invertebrate and vertebrate genomes. egl-28 is expressed in neurons, intestine and head muscle of C. elegans. Tissue specific expression and behavioral analysis indicate that EGL-28 functions in neurons to regulate normal behavior as well as sensitivity to amphetamine. Members of the EGL-28 family of putative transporters are physiological targets of amphetamine in C. elegans and other systems.

Materials and Methods

Genetic Selection

Animals were mutagenized with ethylmethanesulfonate (EMS) at 47 mM for 4 hours and allowed to recover to produce F1 progeny. F1s were ruptured in sodium hypochlorite to isolate F2 eggs which were starved in M9 buffer for 16 hours to synchronize worms at the first larval stage. These larvae were plated onto NGM agar plates seeded with an OP50 E. coli bacterial lawn as food (12) and 5 mM d-amphetamine sulfate (Sigma, St Louis, Mo.). In these conditions, the growth of wild-type animals is inhibited, so mutant animals resistant to the growth suppression effects of amphetamine were isolated as gravid adults 3 and 4 days after plating. egl-28(eg814) was the most resistant mutant isolated.

Drug Assays

Pharyngeal contractions were measured by real time observations under a dissecting microscope using a manual counter. In each experiment the pumping rate of 10 animals was counted for each concentration and the figures shown are individual assays representative of multiple experiments. The chronic effect of amphetamine on pharyngeal pumping was assayed on agar plates seeded with bacteria and 5 mM amphetamine. Animals were plated as first day adult and their rate of pharyngeal contractions was counted 20 hours later. Acute amphetamine assays were done by incubating worms for 90 minutes in M9 buffer. Animals were then transferred to agar plates with food and no amphetamine and allowed to recover for 5-10 minutes before counting pumping. For rescue experiments in 20 mM liquid treatment, animals were allowed to recover for 10-45 minutes, but always the same amount of time for every strain. Muscimol experiments were performed on plates with food which were dried one hour before adding muscimol dropwise to final concentrations of 0.1 and 1 mM. Assays for number of eggs held in utero were performed by bleaching 12 animals per strain in individual wells; and the figures shown are representative of multiple experiments.

Genetic Mapping

The physical map position of egl-28(eg814) was identified by outcrossing to the wild strain CB4856 and selecting 945 F2 recombinants based upon a recessive egg-laying defective (Egl-d) phenotype observed in egl-28. Standard single nucleotide polymorphism (SNP) analysis was performed by PCR amplification of fragments containing SNPs in restriction enzyme recognition sites previously identified on Wormbase (Sanger Center, Cambridge, UK). Cosmids and YACs spanning this region were obtained from Alan Coulson (Sanger Center, Cambridge, UK), and microinjected into egl-28(eg814) using standard techniques (13). RNA mediated interference was performed using the Ahringer laboratory library of RNAi bacterial strains and feeding protocols as previously described (14). Complementation tests were performed with egl-28, egl-37 and egl-43 mutants obtained from the Caenorhabditis Genetics Center (CGC, University of Minnesota). Identification of W07A12.7 as egl-28 was verified by sequencing of coding exons and finding mutations within PCR generated fragments from eg814 and n570 alleles.

Bioinformatics

Several full-length cDNAs encoding EGL-28 have been isolated and sequenced by the Kohara group (National Institute of Genetics, Mishima, Japan). These sequences were used to design primers to isolate egl-28 cDNA by PCR and to predict protein sequence for structural analysis. Argos transmembrane predictions and hydrophobicity plots were done with MacVector software (Oxford Molecular Group, plc). These results were combined with Simple Modular Architecture Research Tool (SMART; EMBL, Heidelberg) analysis and subjectively with the hydrophobicity index of individual amino acids to generate best consensus predictions for transmembrane domains. BLAST searches were performed on NCBI and Sanger Center genomic databases. Phylogenetic analyses was done using MacVector and full-length protein sequences as predicted from identified ESTs and genomic sequences. Graphs and statistics were generated using the Microsoft Excel program.

Transgenic Constructs

All transformed lines were generated by microinjection of PCR products. The rescuing genomic fragment from the egl-28 gene was generated by PCR using the following primers: #1: GTGCCAGGTATTTTCTCTGCG and TCAGTCCATCACACTCCTCC. These rescued lines contained a neuronal GFP co-injection marker (15). The GFP transcriptional construct was generated by a standard fusion PCR reaction (16) using primer #1 above and AGTCGACCTGCAGGCATGCAAGCTGCTGAGTCGTTCCATGATTATTAG. The full-length egl-28 cDNA was isolated by PCR using the primers #4:ATGTCATCATCGCCACACACTCAC and #5:TTTATTGTGCTTATCGTTACCCG from an oligodT primed cDNA library extracted from mixed populations of worms (gift of Hongkyun Kim). This fragment was fused to promoters for egl-28 using primer #1 and #6:GTGTGAGTGTGTGGCGATGATGACATATATGTCTGAAGAAAGAAGAAGG, elt-2 using primers #7:CTGAGCTACGGCGATACGAGG and #8:GTGAGTGTGTGGCGATGATGACATTCTATAATCTATTTTCTAGTTTC, and unc-119 using #9:CACCTGAGACGGGAAGGTTGCCG and #10:GTGAGTGTGTGGCGATGATGACATATATGCTGTTGTAGCTGAAAATTTTGGGG. The P_{egl-28}::egl-28 cDNA construct was fused to GFP using primer #1 and #11:AGTCGACCTGCAGGCATGCAAGCTCCACCCTTCTTTGAAATACATTGC. Expression analysis employed an Axioskop microscope (Leica. Germany) and photographs were taken using an ORCA-ER digital camera (Hamamatsu, Japan) with Openlab software (Improvision Inc., Lexington, Mass.).

Results

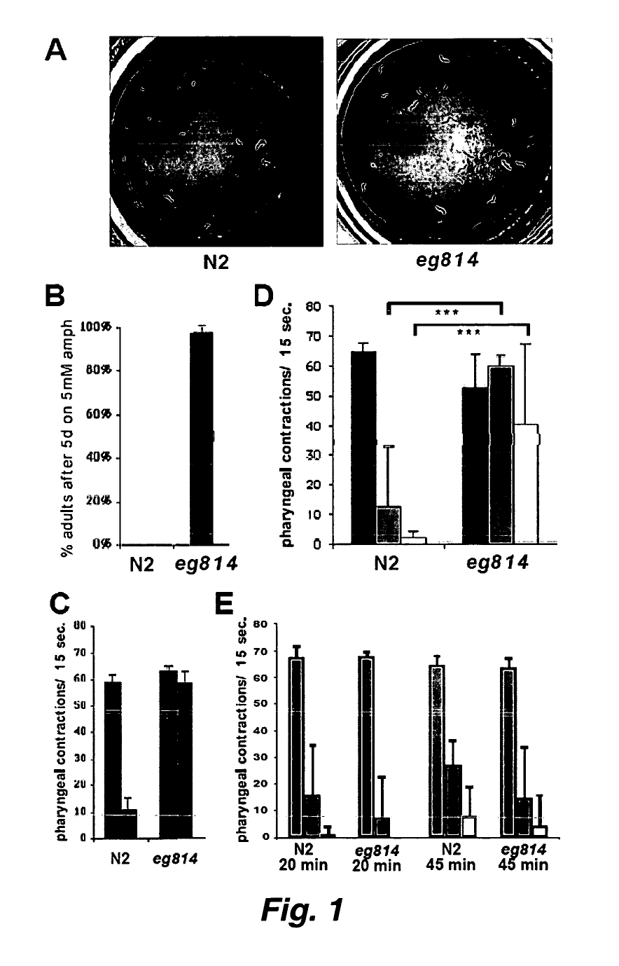

To determine the effects of long-term exposure to amphetamine on the behavior of C. elegans, animals were grown on agar plates prepared with the addition of increasing amounts of amphetamine. Millimolar levels of amphetamine dramatically suppressed the growth of worms. While untreated wild-type (N2) animals are gravid adults after 3 days, on 5 mM amphetamine plates no N2 worms have reached adulthood even after 5 days (FIG. 1A). This observation suggested that a facile genetic selection could be employed to isolate mutant worms resistant to the growth suppressing effects of amphetamine. A genetic selection for C. elegans mutants which show resistance to the growth suppressing effects of amphetamine was performed as described in the methods section.

One strongly resistant mutant, eg814, was isolated which exhibited unaffected growth rates on 5 mM amphetamine. eg814 animals are nearly all gravid adults after 3 days, while no N2 animals have reached this stage (FIGS. 1A and B). The growth suppression effects of chronic amphetamine could possibly have been due to a general toxicity of the drug. A study was therefore carried out to determine if more specific behaviors were altered in eg814. C. elegans draws in and grinds up bacteria by rhythmic contractions of pharyngeal muscles; these contractions can be readily observed and quantified. Mutant and wild-type strains were assayed by transferring first day adults to 5 mM amphetamine plates for 20 hours before quantifying rates of pharyngeal contractions. Following a 20 hour incubation with amphetamine, N2 animals exhibited a greatly reduced rate of pharyngeal contractions (FIG. 1C); however, eg814 animals showed a sustained resistance to the inhibition of pumping (FIG. 1C). The acute effects of amphetamine on pharyngeal pumping were assayed, with the result that increasing amounts of amphetamine inhibits pharyngeal contractions of N2 worms in a dose-dependent manner, while eg814 exhibits resistance to acute amphetamine (FIG. 1D).

Taken together, these data indicate that the resistance to growth suppression exhibited by eg814 may be mediated by a strong resistance to the inhibition of pharyngeal contractions seen in wild-type strains. However, untreated egl-28 animals pump normally. An alternative mechanism is that eg814 animals might be more resistant due to a general impermeability to exogenous drugs. To test this, the effects of the GABAergic agonist muscimol on the rate of pharyngeal contractions(17) was assayed. Increasing amounts of muscimol inhibited pumping in both N2 and eg814 worms with a very similar dose response (FIG. 1E). This indicates that the eg814 resistance to amphetamine is not due to a non-specific impermeability to exogenous drugs or to hyperactive pumping that can not be suppressed by inhibitory neurotransmitters. Overall, this indicated that genetic mapping and molecular characterization of this mutant would give insights into the mechanism of action of amphetamine.

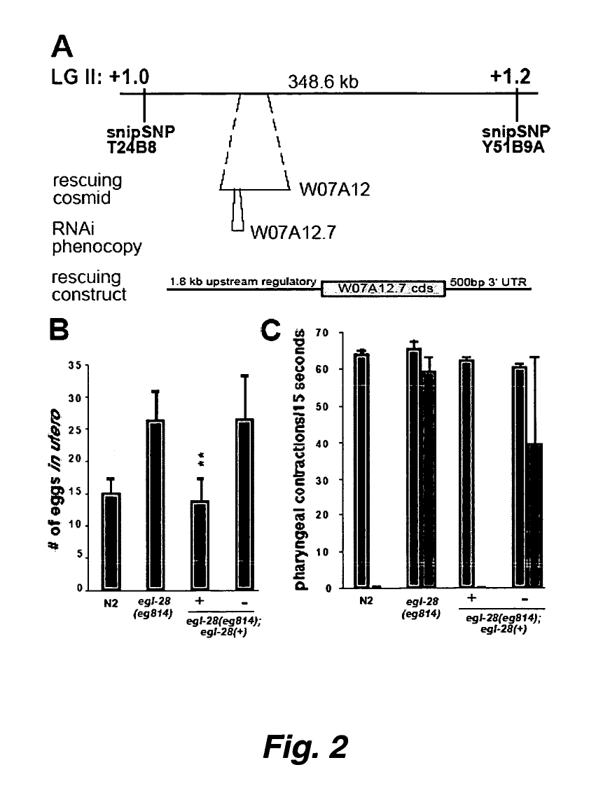

In addition to amphetamine resistance, eg814 animals exhibit an egg-laying defective (Egl-d) phenotype characterized by laying of late-stage eggs and severe bloating due to carrying many more eggs than wild-type worms. This phenotype was used to identify the mutant gene by single nucleotide polymorphism (SNP) mapping. A wild C. elegans strain CB4856 which has previously documented SNPs throughout the six chromosomes was utilized for genetic outcrossing to eg814. Analysis of 945 recombinants revealed that eg814 contained a mutation within an approximately 350 kilobase region near the center of chromosome II (FIG. 2A). Complementation tests were performed with three previously described egl mutants which map within this interval; and it was determined that eg814 fails to complement egl-28(n570)(18) for the Egl-d phenotype. Additionally, n570 animals were resistant to the growth suppression effects of amphetamine (not shown). Microinjection of cosmids spanning this region revealed a single cosmid (W07A12) rescued the egl-28(eg814) Egl-d phenotype (FIG. 2A). Also RNA interference experiments with fragments from 65 coding regions within this genetic interval confirmed that disruption of only the W07A12.7 open reading frame resulted in a severe bloating Egl-d phenotype with virtually 100% penetrance. This indicates that the egl-28 egg-laying defect is due to a loss-function mutation. W07A12.7 is predicted to encode a 502 amino acid multi-pass transmembrane protein with homology to the previously characterized fluoxetine resistant mutants nrf-6 and ndg-4 (19, 20).

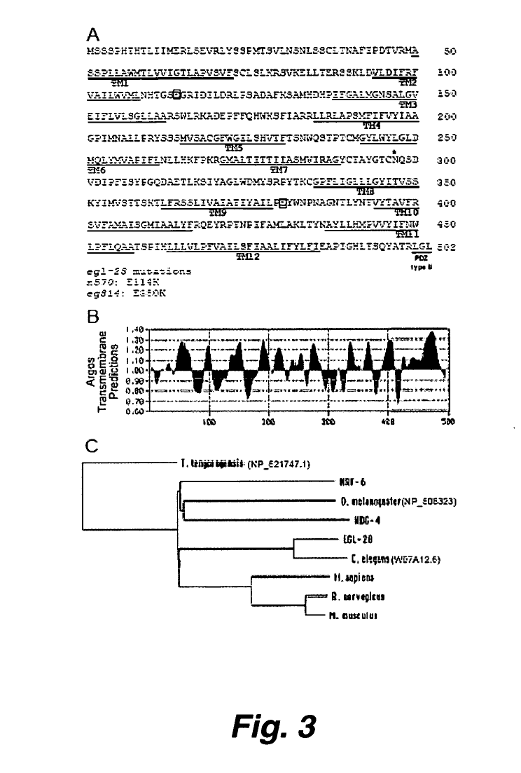

The egl-28(eg814) mutant phenotypes were rescued using a 4.3 kilobase genomic fragment containing W07A12.7 (FIG. 2A). Transformed lines were rescued for the egg-laying defect (FIG. 2B) and also the inhibitory effects of amphetamine on pharyngeal pumping (FIG. 2C). The four exons of the egl-28 coding region isolated from animals carrying the eg814 and n570 alleles were sequenced and single missense mutations were found in each strain (FIG. 3A). Both mutations are guanine to adenosine transitions resulting in substitution of a lysine residue for wild-type glutamate at two different amino acids (FIG. 3A). As shown in FIG. 3A, these are predicted to lie in the first intracellular loop (n570:E114K) and the fifth extracellular loop (eg814: E380K). Predictions of transmembrane spanning segments are a composite of Simple Modular Architecture Research Tool (SMART; EMBL, Heidelberg) analysis, hydrophobicity plots and Argos transmembrane predictions (FIG. 3B). The protein predicted from the sequenced open-reading frame contains no apparent signal sequence, twelve transmembrane spanning segments and therefore intracellular N— and C-termini. A single N-linked glycosylation site and a candidate type II PDZ domain binding motif (21) are predicted (FIG. 3A).

Blast searches of the C. elegans genome reveal that egl-28 is a member of a family of approximately 65 proteins (data not shown), of which the only previously described members were identified in a screen for resistance to the nose contracting effects of high levels of fluoxetine (prozac)(19). This family can be subdivided into three classes, those which contain an N-terminal extension of about 300 amino acids (NRF domain), a C-terminal extension of similar length, and a class including EGL-28 which contains no N— or C-terminal extensions. Further searches indicate that the human, mouse and rat genomes all contain a single related protein with an N-terminal extension (FIG. 3C). This suggests that an ancestral protein was duplicated many times in worms to give rise to the large family found today. In fact, similar to recent duplications observed for several other family members in C. elegans , egl-28 has a very close homologue (W07A12.6) 3.5 kb upstream in the genome, which is 90% identical at the amino acid level. Large families of related proteins are also found in Drosophila and Anopheles, suggesting that they may have some conserved function important for invertebrate physiology.

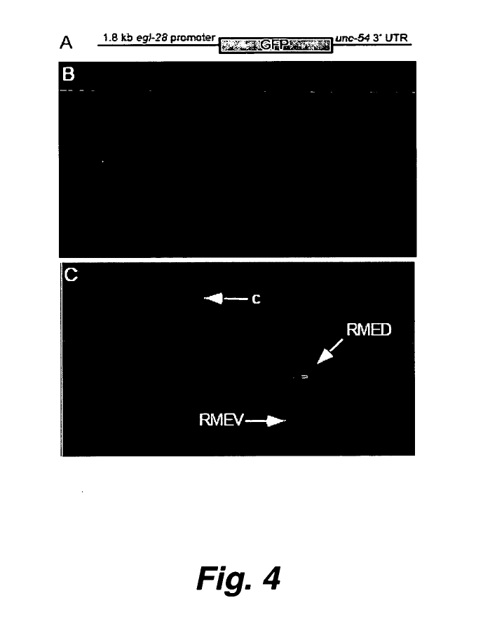

To elucidate the expression pattern of egl-28, wild-type worms were transformed with GFP reporter constructs composed of fusions of egl-28 regulatory regions to green fluorescent protein (GFP). The transcriptional fusion construct shown in FIG. 4A shows GFP expression in the anterior most head muscle cells in all four quadrants (FIG. 4B); muscle arms projecting from these muscle cells are also clearly labeled (not shown). Two neurons in the head express egl-28, which we identified as the RMED and RMEV motorneurons (FIG. 4C). Additional unidentified neurons in the tail occasionally express lower levels of egl-28 (not shown). Intestinal cells are labeled throughout, with the strongest expression at the anterior and posterior ends of the gut (not shown). In animals transformed with a translational fusion construct containing egl-28 coding sequence fused to GFP, RMED and RMEV posterior projections were labeled in a punctate pattern (not shown).

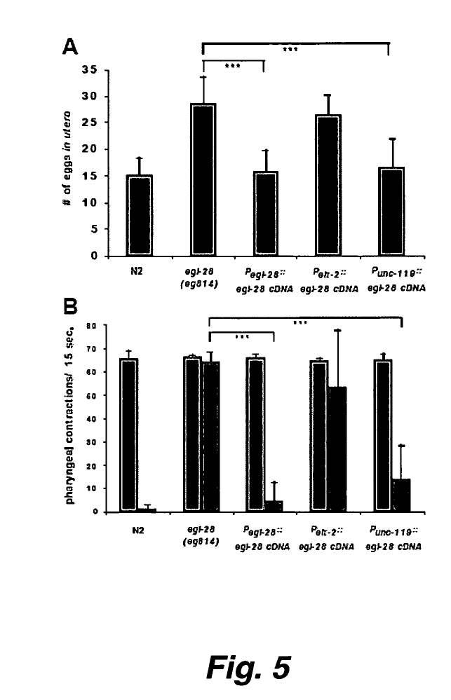

Finally, studies were carried out to determine in which cell type egl-28 was required for normal egg-laying and responsiveness to amphetamine. Although egl-28 mutants are not resistant to all exogenous compounds, it remained possible that EGL-28 could function as a transporter allowing amphetamine to enter the worm, probably through the gut. To test this we transformed egl-28(eg814) animals with constructs expressing egl-28 cDNA under the control of upstream regulatory regions of egl-28 as well as tissue specific promoters for intestine (Pₑₗₜ₋₂)(22) and neurons (P_{unc-119})(23) and compared these lines to wild-type and egl-28 mutants. While expression of egl-28 cDNA in intestine had no effect upon the egg-laying phenotype of egl-28 animals, expression in neurons using the unc-119 promoter restores normal egg-laying behavior (FIG. 5A). Responsiveness to the acute effects of amphetamine was also rescued by the egl-28 endogenous promoter as well as neuronal expression with the unc-119 promoter (FIG. 5B). Again, expression of egl-28 with the elt-2 promoter was not sufficient to restore responsiveness to amphetamine (FIG. 5B). Together, this indicates that EGL-28 functions in neurons to regulate egg-laying and mediate responsiveness to amphetamine.

Discussion

A novel genetic selection in C. elegans was employed to identify important neuronal targets of amphetamine. One isolated mutant exhibited strong resistance to the effects of amphetamine on growth and inhibition of feeding behavior. The mutant also exhibited a baseline defect in egg laying behavior. By mapping and complementation testing, it was found that the amphetamine resistant mutant is allelic to egl-28 (18). egl-28 encodes a protein with twelve transmembrane domains and is a member of the nrf-6, ndg-4 family of putative transporters (19). The EGL-28 protein family includes representative members in other invertebrates and vertebrates including humans. The only previously characterized members (nrf-6 and ndg-4) of this family in any species were found in C. elegans by screening for animals resistant to the nose-contracting effects of fluoxetine (19), another inhibitor of monoamine uptake. Fluoxetine has been shown to bind to and block the reuptake of serotonin by a twelve-membrane spanning transporter (SERT)(24). Amphetamine is known to inhibit both SERT and the related dopamine transporter, DAT (25). SERT and DAT are well characterized in C. elegans (26, 27) and in vertebrates and are distinct from the egl-28, nrf-6, ndg-4 family of putative transporters. It may be that EGL-28 and related proteins act as transporters of other neuromodulators and represent additional targets of amphetamine or fluoxetine in both C. elegans and vertebrates.

It is known that amphetamine has binding sites and mechanisms of action that cannot be easily explained by inhibition of reuptake of dopamine and serotonin. DAT knockout animals still self-administer cocaine (6) and exhibit a paradoxical slowing of locomotion when given amphetamine (9). Binding studies suggest multiple additional possible sites of action in brain (28). For instance, amphetamine binding sites are present in hypothalamus which are not readily displaced by dopamine or other aminergic agonists (11) and this binding affinity correlates well with anorexic effects of amphetamine related compounds but not hyperactivity inducing effects. Identifying additional target molecules is important to understanding the different behavioral effects and neurotoxic effects of amphetamine and related neurostimulants. Psychostimulants have been widely used in the treatment of ADHD, obesity and respiratory ailments but are limited by neurotoxic effects. Through a better understanding of the mechanisms of action, it will be possible to identify more specific compounds that retain the therapeutic benefits without the unwanted side effects.

Although egl-28, nrf-6 and ndg-4 are members of the same family, only egl-28 is expressed in the nervous system of C. elegans. nrf-6 and ndg-4 were found to be expressed in intestinal and hypodermal cells (19). It was postulated that these putative transporters may mediate uptake of fluoxetine into the worm. nrf-6 and ndg-4 might work as a heterodimeric channel or in series on the apical and lumenal surface of intestinal or hypodermal cells as portal of entry for fluoxetine. Although egl-28 is also expressed in the intestine, the results discussed above indicate that intestinal expression of egl-28 is insufficient to restore behavioral sensitivity to amphetamine (inhibition of feeding) and insufficient to rescue the behavioral defect in egg-laying. Instead, it was found that expression of egl-28 cDNA in neurons under the control of the pan-neuronal promoter unc-119 restored normal egg-laying and responsiveness to amphetamine. This is the first evidence that a member of this class of putative transporters functions in the regulation of neuronal activity. EGL-28 may nevertheless have a related function in the intestine. It should be noted that there are many examples of gastrointestinal peptides with widespread activities, suggesting that neuronal and intestinal actions are not incompatible. And it is seems reasonable that transporters for amino acid neurotransmitters could have evolved from ancestral transporters for peptides taken up by the gut.

Like other members of this family, the endogenous substrate of EGL-28 has not been identified, but it appears to function in the neural regulation of egg-laying and in amphetamine mediated inhibition of feeding behavior. It is possible that a neurotransmitter or neuromodulator recognized by EGL-28 functions at synapses between the RMEs and other neurons. RMEs have connections to the RIP neurons, which are the only extrapharyngeal neurons that make contact with the pharyngeal nervous system. The RME neurons are known to be GABAergic (29), but may also release an additional neuromodulator. It is unlikely that EGL-28 transports GABA because a GAT related GABA transporter has been identified, and egl-28 mutants do not exhibit any of the characteristic defects resulting from impaired GABAergic function in C. elegans (17). The substrate recognized by EGL-28 may alternatively function as a neurohumoral factor. It is noteworthy that although egl-28 is required for normal egg-laying behavior, it is not expressed in the egg-laying muscles or neurons that are known to be directly involved in egg-laying.

In view of the foregoing, EGL-28-like proteins may have an enzymatic activity acting on neurotransmitters and/or may act as a transporter at the blood-brain barrier to facilitate access of hydrophobic molecules to the brain.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Hardman, J. G and Limbird, L. E. (2001) The Pharmacological Basis of Therapeutics (McGraw Hill).

2. Brown, J. M. & Yamamoto, B. K. (2003) Pharmacol Ther 99,45-53.

3. Everitt, B. J. & Wolf, M. E. (2002) J Neurosci 22, 3312-20.

4. Sora, I., Hall, F. S., Andrews, A. M., Itokawa, M., Li, X. F., Wei, H. B., Wichems, C., Lesch, K. P., Murphy, D. L. & Uhl, G. R. (2001) Proc Natl Acad Sci USA 98, 5300-5.

5. Shi, W. X., Pun, C. L., Zhang, X. X., Jones, M. D. & Bunney, B. S. (2000) J Neurosci 20, 3504-11.

6. Rocha, B. A., Fumagalli, F., Gainetdinov, R. R., Jones, S. R., Ator, R., Giros, B., Miller, G. W. & Caron, M. G. (1998) Nat Neurosci 1, 132-7.

7. Giros, B., Jaber, M., Jones, S. R., Wightman, R. M. & Caron, M. G. (1996) Nature 379, 606-12.

8. Berke, J. D. & Hyman, S. E. (2000) Neuron 25, 515-32.

9. Gainetdinov, R. R., Mohn, A. R., Bohn, L. M. & Caron, M. G. (2001) Proc Natl Acad Sci USA 98, 11047-54.

10. Gainetdinov, R. R., Wetsel, W. C., Jones, S. R., Levin, E. D., Jaber, M. & Caron, M. G. (1999) Science 283, 397-401.

11. Paul, S. M., Hulihan-Giblin, B. & Skolnick, P. (1982) Science 218, 487-90.

12. Brenner, S. (1974) Genetics 77, 71-94.

13. Mello, C. & Fire, A. (1995) Methods Cell Biol 48, 451-82.

14. Kamath, R. S. & Ahringer, J. (2003) Methods 30, 313-21.

15. Shioi, G., Shoji, M., Nakamura, M., Ishihara, T., Katsura, I., Fujisawa, H. & Takagi, S. (2001) Genetics 157, 1611-22.

16. Hobert, 0. (2002) Biotechniques 32, 728-30.

17. McIntire, S. L., Jorgensen, E. & Horvitz, H. R. (1993) Nature 364, 334-7.

18. Trent, C., Tsuing, N. & Horvitz, H. R. (1983) Genetics 104, 619-47.

19. Choy, R. K. & Thomas, J. H. (1999) Mol Cell 4, 143-52.

20. Shreffler, W., Magardino, T., Shekdar, K. & Wolinsky, E. (1995) Genetics 139, 1261-72.

21. Hung, A. Y. & Sheng, M. (2002) J Biol Chem 277, 5699-702.

22. Hawkins, M. G. & McGhee, J. D. (1995) J Biol Chem 270, 14666-71.

23. Maduro, M. & Pilgrim, D. (1995) Genetics 141, 977-88.

24. Sanchez, C. & Hyttel, J. (1999) Cell Mol Neurobiol 19, 467-89.

25. Sonders, M. S., Zhu, S. J., Zahniser, N. R., Kavanaugh, M. P. & Amara, S. G. (1997) J Neurosci 17, 960-74.

26. Ranganathan, R., Sawin, E. R., Trent, C. & Horvitz, H. R. (2001) J Neurosci 21, 5871-84.

27. Jayanthi, L. D., Apparsundaram, S., Malone, M. D., Ward, E., Miller, D. M., Eppler, M. & Blakely, R. D. (1998) Mol Pharmacol 54, 601-9.

28. Hauger, R. L., Hulihan-Giblin, B., Skolnick, P. & Paul, S. M. (1984) Life Sci 34, 771-82.

29. McIntire, S. L., Jorgensen, E., Kaplan, J. & Horvitz, H. R. (1993) Nature 364, 337-41.

1. A method of modulating a response to a neurotransmitter or a psychostimulant, the method comprising contacting cells expressing a member of the egl-28 gene family, with an effective amount of a modulator of said member of the egl-28 gene family, in the presence of the neurotransmitter or psychostimulant, respectively.

2. The method of claim 1, wherein the neurotransmitter is histamine, and the psychostimulant is amphetamine.

3. The method of claim 2, wherein the modulator comprises an inhibitor of the member of the egl-28 gene family.

4. The method of claim 2, wherein the member of the egl-28 gene family comprises a polypeptide including an amino acid sequence that has at least about 50% identity to the C. elegans egl-28 amino acid sequence (SEQ ID NO:3) over a comparison window of at least 17 contiguous amino acids.

5. The method of claim 4, wherein the amino acid sequence has at least about 20% identity to the full-length the C. elegans egl-28 amino acid sequence (SEQ ID NO:3).

6. The method of claim 2, wherein the cells are in vitro.

7. An isolated polypeptide comprising an amino acid sequence, wherein the amino acid sequence has at least about 50% identity to the C. elegans egl-28 amino acid sequence (SEQ ID NO:3) over a comparison window of at least 17 contiguous amino acids, provided said polypeptide is not nfr-6 or ndg-4.

8-14. (canceled)

15. An isolated polynucleotide that encodes the polypeptide of claim 7.

16-22. (canceled)

23. An antibody or antiserum that specifically binds to the polypeptide of claim 7.

24. (canceled)

25. A method of identifying an egl-28 ortholog, said method comprising determining whether a candidate egl-28 ortholog polynucleotide comprises a nucleotide sequence that is substantially similar to an egl-28 nucleotide sequence and/or whether a candidate egl-28 ortholog polypeptide comprises an amino acid sequence that is substantially similar to an egl-28 amino acid sequence.

26-32. (canceled)

33. A method of prescreening for an agent that modulates a response to a neurotransmitter or a psychostimulant, the method comprising:
a) contacting a test agent with a polypeptide encoded by a member of the egl-28 gene family or a polynucleotide encoding said polypeptide; and
b) detecting specific binding of the test agent to the polypeptide or polynucleotide.

34-38. (canceled)

39. A method of screening for an agent that modulates a response to a neurotransmitter or a psychostimulant, said method comprising:
a) contacting a test agent with a cell that expresses a member of the egl-28 gene family; and
b) determining the level of:
(i) the polypeptide encoded by said member of the egl-28 gene family;
(ii) the RNA transcribed from said member of the egl-28 gene family; or
(iii) a neurotransmitter- or a psychostimulant-induced response mediated by said member of the egl-28 gene family.

40-50. (canceled)


SEQUENCE LISTING (17 sequences)

SEQ ID NO: 1 - RNA, 14 nt, Artificial
  hairpin ribozyme
  misc_feature (1)..(3): N is any of G, U, C, or A
  misc_feature (4)..(4): N is any of G, C, or U
  misc_feature (5)..(5): N is any of G, U, C, or A
  misc_feature (9)..(14): N is any of G, U, C, or A
nnnnngucnnnnnn

SEQ ID NO: 2 - DNA, 1509 bp, Caenorhabditis elegans
atgtcatcatcgccacacactcacactctaataatcatggaacgactcagcgaagtgcgattgtactcatcaccaatgacatcagtactcaactcaaatctatcttcttgcttaacaaatgcatttattcctgatactgtacgaatggccagtagtccacttttggcatggatgacacttgtcgtcatcggaacacttgctccagtaagcgtcttttcctgcttgagcctcaagcgatcagtcaaagaattgctcactgaaagatcctcgaaactcgatgtattggatatcttccgattcgttgcaatcctttgggttatgcttaaccatactggaagtgaaggaagaattgatattttggatcgactaccatctgctgatgcattcaaaagtgcaatgcatgatcatccaatttttggagctctcatgggaaactctgctcttggagttgaaattttccttgtactttctggacttttggcagctagatcgtggcttcgtaaagccgatgagccatttttccaacattggaaatcatttattgctcgtagactactccgcttggctccatccatgttcatttttgtctacatcgctgctggtccaatcatgaatgctcttctcccacgatactcctcttcaatggtctccgcttgtggtttttggggtattctttcccatgtaacattcacttctaattggcaatccacaccaacttgcatgggatatctttggtatttgggactcgacatgcaactttacatggttgctccaatcttcttgaatcttcttcacaagtttccaaaacgtggaatggctctcactattaccactataattgcctctatggtcatccgtgcgggttactgtaccgcctatggaacttgcaaccaaagtgatgtcgatattccattcatttcatatccagggcaagacgcagagacattgaagagtatttatgctggactttgggacatgtactcaagaccatataccaagtgtggtccattccttatcggtcttcttctcggatacatcacagtttccagcaaatacatcatggtttcgactacatccaagacactcttccgttccagtctcattgtcgcaatcgccacaatctacgcaattctcccagaatattggaacccaaatgctggaaatactctctataatacagtttacacggcagtattccgatctgtattcgctatggctatttctggaatgattgctgctctgtatttcagacaagaataccgtccaacaaatccgatcttcgccatgcttgccaagctcacctacaacgcgtatctccttcatatgccagttgtctatattttcaattggctcccattccttcaagctgccacttcaccaattcatcttctactagttcttccatttgttgcaattctatcattcattgctgctctcatcttttatcttttcattgaggccccaattgggcatttgacatctcaatatgccacacgactgggcctgtag

SEQ ID NO: 3 - protein, 502 aa, Caenorhabditis elegans
MSSSPHTHTLIIMERLSEVRLYSSPMTSVLNSNLSSCLTNAFIPDTVRMASSPLLAWMTLVVIGTLAPVSVFSCLSLKRSVKELLTERSSKLDVLDIFRFVAILWVMLNHTGSEGRIDILDRLPSADAFKSAMHDHPIFGALMGNSALGVEIFLVLSGLLAARSWLRKADEPFFQHWKSFIARRLLRLAPSMFIFVYIAAGPIMNALLPRYSSSMVSACGFWGILSHVTFTSNWQSTPTCMGYLWYLGLDMQLYMVAPIFLNLLHKFPKRGMALTITTIIASMVIRAGYCTAYGTCNQSDVDIPFISYPGQDAETLKSIYAGLWDMYSRPYTKCGPFLIGLLLGYITVSSKYIMVSTTSKTLFRSSLIVAIATIYAILPEYWNPNAGNTLYNTVYTAVFRSVFAMAISGMIAALYFRQEYRPTNPIFAMLAKLTYNAYLLHMPVVYIFNWLPFLQAATSPIHLLLVLPFVAILSFIAALIFYLFIEAPIGHLTSQYATRLGL

SEQ ID NO: 4 - DNA, 2115 bp, Homo sapiens
atgcgtatatgttatgaatgccaaaatgaaagaacattgtggcgatgtgtttcccaggatggggctgactacagtgtgggcgtgtgtgtccctgattcttgtgctgaagaggatgtgactctgatgtctcggctggatactttaagattcagaaatacttcatttttggccccttccctctttctttttacaataaattcttcctccttgtctggtgggagtgtgaccagatgtgctgctggaaagatccccctggacacatttgctgccgtatgtctgttcatcaccttgctgggtctcatcctccctccggctggaacagtctgcgtggcagctagggaatgggggtcagcctgcaggacatcgcgggaacacggggaacctctggccacttacgggagtctgccactgagcgaggcggagagcaatgaacaaagaagcagaatcccacggacacactgccgggcacatctcctcctgtcagcagcctccagcagaggaaaaaggtttctaggagccgtggctcatgctctggagtgcttttcttggcagaagaatgtgccagccatctggactacaaaggcaccaggtggcacctgctctgcactgaatggcattcgtgtcttgagtcttctttggatcatctcgggacacaccagtcagatgactgcatggctgtctttgggatggaaagatggagggcacgaaaggccactggtcatgtctgggccatcagtgggaatcggagacaccagagaagccacgagtggttggttaagtgcaagttcgtttttaaagatgcatcagaattcagacaaaggaataacccccaaaggcatactcagatactttctcagtcacctggtaaggttgcagcctcttcacctgtattcaatgtgcttgttggttggactgttctctcttgttccctggggacctgtctgggaaatgcccaaattccactgggataactgccggcaagcatggtggacgaatctgctgttgctaaataactttgtgtcggtcaagaatgcgtgcaatggctggacctggtaccttgccaatgacttccagttccacctcaccacaccagtgattatcttcatccatgtaaagagtacacagatcctcatcctccttggggccatgctgttcttggcatctttcacagccactgctctgatcaccttggcatataaacttcctgtcgtggctccatcagaaaccaggacttcccggggagggctgctgaatgccaggctgttcaccctgtgccctttggttcatggaaaaagtgggtatgaaacttttggtctggatgggaaagctgattgccttcttgcttccaaacttctgaacctttcaacctgcactggaaatgaacaagtgtgccctaaatgtacctttgggcttgctgattattccaatggacatctcagggatttggattccctttgccatgtccagatcaaacataacattttggcttatttccttgtatttttcagtgaagaggcgattgtattgtatttcgtggagtactacacaaagccctactgccgatttgggccagttcttgtgggcctctttctgagcatttacatgcaccaaaaccaccaggaaaacattctcagaaccaagctgcagctctctaccaagccctccaccggaccctgtgggcggcggctgtgggctgagtcctctttgcgtgccacggaggatatggaggtatggaagcggctccaggctttgctgtcgggttcacaccctgttcctttaaaggtgacaaatcgaacacacaggagagccaagcagataaaaggcttcaatggaaaagaatcttctccaggtctggtgaaccgtgtgctttcttgggacatctggagtttcctgtccagcatcagttatgctcgctacttggtgcatccgattctgatcatcctttacaatggccttcaggaaacacttattcaccacactgacaccaacatgttctatcttttctctggacaccgtgtgctgaccttcgtcactgggctggccctgacgctgttcattgagaaaccatgtcaggaactgaagcagcacctgctgggccatgaatgttctggttaa

SEQ ID NO: 5 - protein, 704 aa, Homo sapiens
MRICYECQNERTLWRCVSQDGADYSVGVCVPDSCAEEDVTLMSRLDTLRFRNTSFLAPSLFLFTINSSSLSGGSVTRCAAGKIPLDTFAAVCLFITLLGLILPPAGTVCVAAREWGSACRTSREHGEPLATYGSLPLSEAESNEQRSRIPRTHCRAHLLLSAASSRGKRFLGAVAHALECFSWQKNVPAIWTTKAPGGTCSALNGIRVLSLLWIISGHTSQMTAWLSLGWKDGGHERPLVMSGPSVGIGDTREATSGWLSASSFLKMHQNSDKGITPKGILRYFLSHLVRLQPLHLYSMCLLVGLFSLVPWGPVWEMPKFHWDNCRQAWWTNLLLLNNFVSVKNACNGWTWYLANDFQFHLTTPVIIFIHVKSTQILILLGAMLFLASFTATALITLAYKLPVVAPSETRTSRGGLLNARLFTLCPLVHGKSGYETFGLDGKADCLLASKLLNLSTCTGNEQVCPKCTFGLADYSNGHLRDLDSLCHVQIKHNILAYFLVFFSEEAIVLYFVEYYTKPYCRFGPVLVGLFLSIYMHQNHQENILRTKLQLSTKPSTGPCGRRLWAESSLRATEDMEVWKRLQALLSGSHPVPLKVTNRTHRRAKQIKGFNGKESSPGLVNRVLSWDIWSFLSSISYARYLVHPILIILYNGLQETLIHHTDTNMFYLFSGHRVLTFVTGLALTLFIEKPCQELKQHLLGHECSG

SEQ ID NO: 6 - skipped

SEQ ID NO: 7 - DNA, 21 bp, Artificial
  oligonucleotide primer
gtgccaggtattttctctgcg

SEQ ID NO: 8 - DNA, 20 bp, Artificial
  oligonucleotide primer
tcagtccatcacactcctcc

SEQ ID NO: 9 - DNA, 48 bp, Artificial
  oligonucleotide primer
agtcgacctgcaggcatgcaagctgctgagtcgttccatgattattag

SEQ ID NO: 10 - DNA, 24 bp, Artificial
  oligonucleotide primer
atgtcatcatcgccacacactcac

SEQ ID NO: 11 - DNA, 23 bp, Artificial
  oligonucleotide primer
tttattgtgcttatcgttacccg

SEQ ID NO: 12 - DNA, 49 bp, Artificial
  oligonucleotide primer
gtgtgagtgtgtggcgatgatgacatatatgtctgaagaaagaagaagg

SEQ ID NO: 13 - DNA, 21 bp, Artificial
  oligonucleotide primer
ctgagctacggcgatacgagg

SEQ ID NO: 14 - DNA, 47 bp, Artificial
  oligonucleotide primer
gtgagtgtgtggcgatgatgacattctataatctattttctagtttc

SEQ ID NO: 15 - DNA, 23 bp, Artificial
  oligonucleotide primer
cacctgagacgggaaggttgccg

SEQ ID NO: 16 - DNA, 53 bp, Artificial
  oligonucleotide primer
gtgagtgtgtggcgatgatgacatatatgctgttgtagctgaaaattttgggg

SEQ ID NO: 17 - DNA, 48 bp, Artificial
  oligonucleotide primer
agtcgacctgcaggcatgcaagctccacccttctttgaaatacattgc